SEQUENCE LISTING (3 sequences)

SEQ ID NO: 1 - DNA, 2888 bp, Homo sapiens
cgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaaggtaaa

SEQ ID NO: 2 - DNA, 2222 bp, Homo sapiens
gagtcccggcagtgcgggactccggtagccgcccctccggtagccgcccctcctccccccgcgccgccgccctatatgttgcccgccgcggcctctggggcatggagcacgctgcccagccctggcgatggcaacggcgacgggggtggcggaggagcgcctgctggccgcgctcgcctacctgcagtgcgccgtgggctgcgcggtcttcgcgcggaatcgtcagacgaactcagtgtacggccgccacgcgctgcccagccacaggctccgagtgccggcgcgggccgcctgggtggtgcaggagctgccctcgctggccctgccgctctaccagtacgccagcgagtccgccccgcgtctccgcagcgcgcccaactgcatcctcctggccatgttcctcgtccactacgggcatcggtgcttaatttacccgtttctgatgcgaggaggaaagcctatgccactgttggcatgtacaatggcgattatgttctgtacctgtaacggctatttgcaaagcagatacttgagccattgtgcagtgtatgctgatgactgggtaacagatccccgttttctaataggttttggcttgtggttaacaggcatgttgataaacatccattcagatcatatcctaaggaatctcagaaaaccaggagatactggatacaaaataccaaggggaggcttatttgaatacgtaactgcagccaactattttggagaaatcatggagtggtgtggctatgccctggccagctggtctgtccaaggcgcggcttttgctttcttcacgttttgttttttatctggtagagcaaaagagcatcatgagtggtacctccggaaatttgaagagtatccaaagttcagaaaaattataattccatttttgttttaagtgcgtttttcatgaaattatcttcaacttgaagctttccaatggcgcttctctatggactttgtaaataagttatatctttgtaattttcctgctactttatcattttcaagatgtcctctaggaattttttttctagtaattttgcaatctacctaataagtacctaaatacgctgaaatggaggttgaatatcctactgtgtaacaggtcagaatttcaagctctgggtaataactgctgatattttttctaatttcaaatttacctcttttggctatgtcttgccaagtgtgtatgagactagactttacaactgtctttgatggcattttcagaacaataaatgtcacaatcccttctatagccccctacagtgatctcttcaaggtcaactgcagtgttgcttccctccccctatagggctggaatctgtctaggagccctctctcggaggccatagaggctgggggtagccattgtgcagtcatggcccgggggaaacttgccaaccttcgtgtcaggtgctgtgtataagtggagaacttggggatagaggaggaagctcctcgtggcccttccaaggtgaggcaaaggcatctggacttgttccagcccagcccaccgggtgacatcaccgggcagggaggggtgctggtggtggttcatacggagtaagctgctctgcctgtgtgagtggctcctgggccctaaacaggcacctttaggccatgggtcactcaccgtgagccatcaatgtgctctggtctgacatggtttctctctgtcttctagtctagacctagtttttttgttctgttccccacgtatggatatagtagagattgttgtctgtgaaatttctcttttgtagattttgagttttcccttgtagtgtaaagaatgatcactttctgtaacaataacaagaccactttttaagatttatcctgtttgttctttgttgattgaaacataataattgttaaaattctctacagccttctttttcttccatagctaatcttccttctaatagtttttgctttctgttttgctgttgttgctttgcaaagctttcccctcatagcctgtacctgttatcaatataaaataatcttcctgttgaatgcttcatgacttgaattctactttgataaaaacattgccatactgctttttatcttgatgaattcatctggcattgctttgccttatcatctcatctggagtttttaaatgccatttgtttcagttgtctttaacaacataataaatagactttgccatttaaaaaaaaaaaaaaaaaaaaaaa

SEQ ID NO: 3 - DNA, 2481 bp, Homo sapiens
tccataaaggggttgcgggggccgcgctctcttctgggagggcagcggccaccggcgaggaacacggcgcgatgcaggttcagtgccagcagagcccagtgctggcaggcagcgccactttggtcgcccttggggcactggccttgtacgtcgcgaagccctccggctacgggaagcacacggagagcctgaagccggcggctacccgcctgccagcccgcgccgcctggttcctgcaggagctgccttccttcgcggtgcccgcggggatcctcgcccggcagcccctctccctcttcgggccacctgggacggtacttctgggcctcttctgcgtacattacttccacaggacatttgtgtactcactgctcaatcgagggaggccttatccagctatactcattctcagaggcactgccttctgcactggaaatggagtccttcaaggctactatctgatttactgtgctgaataccctgatgggtggtacacagacatacggtttagcttgggtgtcttcttatttattttgggaatgggaataaacattcatagtgactatatattgcgccagctcaggaagcctggagaaatcagctacaggattccacaaggtggcttgtttacgtatgtttctggagccaatttcctcggtgagatcattgaatggatcggctatgccctggccacttggtccctcccagcacttgcatttgcatttttctcactttgtttccttgggctgcgagcttttcaccaccataggttctacctcaagatgtttgaggactaccccaaatctcggaaagcccttattccattcatcttttaaaggaaccaaattaaaaaggagcagagctcccacaatgctgatgaaaactgtcaagctgctgaaactgtaattttcatgatataatagtcatatatatatatatatatatatatatatatatatatatgtatatatgtaatagtaggtctcctggcgttctgccagctggcctggggattctgagtggtgtctgcttagagtttactcctacccttccagggacccctatcctgatccccaactgaagcttcaaaaagccacttttccaaatggcgacagttgcttcttagctattgctctgagaaagtacaaacttctcctatgtctttcaccgggcaatccaagtacatgtggcttcatacccactccctgtcaatgcaggacaactctgtaatcaagaattttttgacttgaaggcagtacttatagaccttattaaaggtatgcattttatacatgtaacagagtagcagaaatttaaactctgaagccacaaagacccagagcaaacccactcccaaatgaaaaccccagtcatggcttcctttttcttggttaattaggaaagatgagaaattattaggtagaccttgaatacaggagccctctcctcatagtgctgaaaagatactgatgcattgacctcatttcaaatttgtgcagtgtcttagttgatgagtgcctctgttttccagaagatttcacaatccccggaaaactggtatggctattcttgaaggccaggttttaataaccacaaacaaaaaggcatgaacctgggtggcttatgagagagtagagaacaacatgaccctggatggctactaagaggatagagaacagttttacaatagacattgcaaactctcatgtttttggaaactggtggcaatatccaaataatgagtagtgtaaaacaaagagaattaatgatgaggttacatgctgcttgcctccaccagatgtccacaacaatatgaagtacagcagaagccccaagcaactttcctttcctggagcttcttccttgtagttctcaggacctgttcaagaaggtgtctcctaggggcagcctgaatgcctccctcaaaggacctgcaggcagagactgaaaattgcagacagaggggcacgtctgggcagaaaacctgttttgtttggctcagacatatagtttttttttttttacaaagtttcaaaaacttaaaaatcaggagattccttcataaaactctagcattctagtttcatttaaaaagttggaggatctgaacatacagagcccacatttccacaccagaactggaactacgtagctagtaagcatttgagtttgcaaactcttgtgaaggggtcaccccagcatgagtgctgagatatggactctctaaggaaggggccgaacgcttgtaattggaatacatggaaatatttgtcttctcaggcctatgtttgcggaatgcattgtcaatatttagcaaactgttttgacaaatgagcaccagtggtactaagcacagaaactcactatataagtcacataggaaacttgaaaggtctgaggatgatgtagattactgaaaaatacaaattgcaatcatataaataagtgtttttgttgttcattaaatacctttaaatcatg

(19) United States
(12) Patent Application Publication
(10) Pub. No.: US 2007/0036740 A1
(43) Pub. Date: Feb. 15, 2007

(54) Modulation of hair growth

(76) Inventor: Kenneth Clifford Reed, Queensland (AU)

Correspondence Address: PATTON BOGGS LLP, 8484 WESTPARK DRIVE, SUITE 900, MCLEAN, VA, 22102, US

(21) Appl. No.: 11/244,314

(22) Filed: Oct. 5, 2005

Related U.S. Application Data
(60) Provisional application No. 60/616,423, filed on Oct. 6, 2004.

(51) Int. Cl. A61K 8/73; A61K 48/00

(52) U.S. Cl. 424/70.13; 514/44

(57) ABSTRACT

The present invention provides methods and compositions including RNAi agents for modulating the growth, development, or maintenance of hair or hair follicles in vertebrate animals.

### CROSS-REFERENCE TO RELATED APPLICATIONS

This application claims benefit of U.S. provisional patent application Ser. No. 60/616,423, filed Oct. 6, 2004, which is herein incorporated by reference.

### BACKGROUND OF THE INVENTION

1. Field of the Invention

The present invention relates generally to methods and RNAi agents for modulating hair growth in vertebrate animal species. More particularly, the present invention provides siRNA and DNA-directed RNAi (ddRNAi)-based methods for silencing one or more transcriptionally active genetic regions which is or are directly or indirectly associated with the modulation of hair or hair follicle growth, development and/or maintenance. The present invention further extends to ddRNAi expression cassettes, vectors, and other genetic constructs useful in modulating the growth, development, or maintenance of hair or hair follicles.

2. Description of the Prior Art

Bibliographic details of references provided in the subject specification are listed at the end of the specification.

Reference to any prior art in this specification is not, and should not be taken as, an acknowledgment or any form of suggestion that this prior art forms part of the common general knowledge in any country.

Utilization of double-stranded RNA to inhibit gene expression in a sequence-specific manner has revolutionized the drug discovery industry. In mammals, RNA interference, or RNAi, is mediated by 15- to 49-nucleotide long, double-stranded RNA molecules referred to as small interfering RNAs (siRNAs). RNAi agents can be synthesized chemically or enzymatically outside of cells and subsequently delivered to cells (see, eg. Fire et al., Nature 391: 806-11, 1998; Tuschl et al. Genes and Dev. 13: 3191-97, 1999; and Elbashir et al. Nature 411: 494-498, 2001) or can be expressed in vivo by an appropriate vector in cells (see, eg., U.S. Pat. No. 6,573,099).

In vivo delivery of unmodified RNAi agents as an effective therapeutic for use in humans faces a number of technical hurdles. First, due to cellular and serum nucleases, the half life of RNA injected in vivo is only about 70 seconds (see, eg., Kurreck, Eur. J. Bioch. 270: 1628-44, 2003). Efforts have been made to increase stability of injected RNA by the use of chemical modifications; however, there are several instances where chemical alterations led to increased cytotoxic effects. In one specific example, cells were intolerant to doses of an RNAi duplex in which every second phosphate was replaced by phosphorothioate (Harborth et al., Antisense Nucleic Acid Drug Rev. 13(2): 83-105, 2003). Other hurdles include providing tissue-specific delivery, as well as being able to deliver the RNAi agents in amounts sufficient to elicit a therapeutic response, but that are not toxic.

Several options are being explored for RNAi delivery, including the use of viral-based and non-viral based vector systems that can infect or otherwise transfect target cells, and deliver and express RNAi molecules in situ. Often, small RNAs are transcribed as short hairpin RNA (shRNA) precursors from a viral or non-viral vector backbone. Once transcribed, the shRNA are processed by the enzyme Dicer into the appropriate active RNAi agents. Viral-based delivery approaches attempt to exploit the targeting properties of viruses to generate tissue specificity and once appropriately targeted, rely upon the endogenous cellular machinery to generate sufficient levels of the RNAi agents to achieve a therapeutically effective dose.

One useful application of RNAi therapeutics is to control inter alia hair growth. Patterns of hair growth in humans arise from, inter alia, the response to androgenic steroid hormones of skin cells that are capable of producing hair follicles. Differential patterns of hair growth which are characteristic of the male and female sexes result largely from differential circulating levels of androgenic steroid hormones. The distribution of androgen-responsive follicular skin cells is largely similar in both sexes. Sexually dimorphic hair patterns reflect the dimorphism in androgen levels.

For a range of social, aesthetic, hygienic and even cultural reasons, it is considered desirable to control the growth and/or development of body hair. One such circumstance is the progressive loss of scalp hair which is characteristic of many adult men, leading to a condition referred to as “male pattern baldness”. Another circumstance is the growth of hair in sites where it is considered to be undesirable, in usually, but not limited to, adult females. Such sites include but are not limited to the face, eyebrows, armpits, legs, arms and pubic region. In adult males, additional sites in which the growth of hair may be considered undesirable include, but are not limited to, ears, nose and trunk. Furthermore, it may also be desirable to encourage regrowth of hair following medical procedures or treatments including both chemotherapy and radiotherapy.

Androgenic steroid hormones are required for the manifestation of both of these divergent phenotypes—scalp hair loss and enhanced body hair. Their post-pubertal manifestation results from significant increases in circulating androgenic steroids that are initiated at puberty and maintained thereafter in both sexes. Their heightened manifestation in males results from the significantly higher levels of circulating androgenic steroids that are characteristic of males, a product of post-pubertal testicular secretions. The effects of androgenic steroid hormones are mediated by their interaction with proteins which are present within cells which are responsive to androgenic steroids. One such protein is known as the androgen receptor or 5α-dihydrotestosterone (DHT) receptor. DHT is a metabolic derivative of the major circulating androgenic steroid hormone testosterone, arising generally from the intracellular conversion of testosterone within androgen-responsive cells by an enzyme known as steroid 5α-reductase (see U.S. Pat. No. 5,422,262).

The present invention provides stable, effective ddRNAi therapeutics and methods for the use thereof to modulate the growth, development and/or maintenance of hair or hair follicles by altering the level of expression of one or more transcriptionally active genetic regions which are directly or indirectly associated with hair growth.

### SUMMARY OF THE INVENTION

The present invention provides a method for modulating the growth, development or maintenance of hair or hair follicles in an animal subject together with RNAi agents for use therewith, genetic constructs which encode RNAi agents and genetically modified cells comprising the genetic constructs. The present invention is predicated, in part, on the application of agents which facilitate gene silencing via RNAi to downregulate or silence one or more transcriptionally active genetic regions which are directly or indirectly associated with the growth, development and/or maintenance of hair or hair follicles. Such transcriptionally active regions are also referred to herein as “hair associated genetic targets” or “HATs”. RNAi-mediated silencing of one or more HATs effects modulation, including promotion or suppression, of the growth, development or maintenance of hair or hair follicles in the subject.

The RNAi agents of the present invention preferably comprise either siRNAs (synthetic RNAs) or DNA-directed RNAs (ddRNAs).

Accordingly, in one aspect, the present invention contemplates a method for modulating the growth, development and/or maintenance of hair or hair follicles in a subject, said method comprising administering to said subject an siRNA comprising a nucleotide sequence which is at least 70% identical to at least part of a nucleotide sequence comprising a HAT or a derivative, ortholog or homolog thereof and which delays, represses or otherwise reduces the expression of the HAT in said subject.

In another aspect, the present invention contemplates a method for modulating the growth, development and/or maintenance of hair or hair follicles in a subject, said method comprising administering to said subject a genetic construct comprising at least one ddRNAi expression cassette which encodes an RNA molecule comprising a nucleotide sequence which is at least 70% identical to at least part of a nucleotide sequence comprising a HAT or a derivative, ortholog or homolog thereof and which delays, represses or otherwise reduces the expression of the HAT in said subject.

As used herein, the term “ddRNAi expression cassette” refers to a nucleic acid sequence which is able to effect transcription to produce an RNAi agent. Preferably, this includes nucleic acid molecules being single or double stranded, partially double stranded, stem-loop and/or panhandle type molecules. Typically, a ddRNAi expression cassette comprises a promoter operably linked to a ddRNAi targeting sequence which in turn is operably linked to a terminator.

In one preferred embodiment, the ddRNAi expression cassette comprises a nucleic acid molecule comprising the general structure (I): 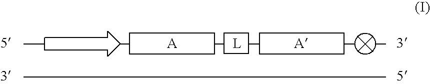
wherein: [symbol]
represents a promoter sequence; [symbol]
represents a ddRNAi targeting sequence comprising at least 10 nucleotides, wherein said sequence is at least 70% identical to a HAT sequence or part thereof; [symbol]
represents a sequence of at least 10 nucleotides wherein at least 10 contiguous nucleotides of A′ comprise a reverse complement of the nucleotide sequence represented by A; [symbol]
represents a “loop” encoding structure comprising a sequence of 5 to 20 non-self-complementary nucleotides; and [symbol]
represents a terminator sequence.

In another preferred embodiment, the ddRNAi expression cassette comprises a nucleic acid molecule of the general structure (II): 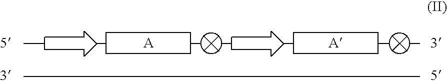
wherein: [symbol]
represents a promoter sequence; [symbol]
represents a ddRNAi targeting sequence comprising at least 10 nucleotides, wherein said sequence is at least 70% identical to a HAT sequence or part thereof; [symbol]
represents a sequence of at least 10 nucleotides wherein at least 10 contiguous nucleotides of A′ comprise a reverse complement of the nucleotide sequence represented by A; and [symbol]
represents a terminator sequence.

In yet another embodiment, the ddRNAi expression cassette comprises a nucleic acid molecule of the general structure (III): 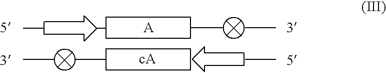
wherein: [symbol]
represents a promoter sequence; [symbol]
represents a ddRNAi targeting sequence comprising at least 10 nucleotides, wherein said sequence is at least 70% identical to a HAT sequence or part thereof; [symbol]
represents a nucleic acid sequence complementary to A; and [symbol]
represents a terminator sequence.

In yet another preferred embodiment, the ddRNAi expression cassette comprises a nucleic acid molecule of the general structure (IV): 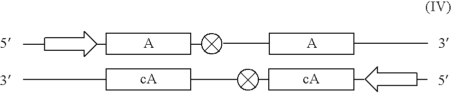
wherein: [symbol]
represents a promoter sequence; [symbol]
represents a ddRNAi targeting sequence comprising at least 10 nucleotides, wherein said sequence is at least 70% identical to a HAT sequence or part thereof; [symbol]
represents a nucleic acid sequence complementary to A; and [symbol]
represents a terminator sequence.

Although the ddRNAi expression cassettes represented by general structures (I), (II), (III) and (IV) represent preferred embodiments of the invention, the present invention is in no way limited to ddRNAi expression cassettes comprising these particular general structures.

In another aspect, the present invention contemplates a ddRNAi expression vector wherein said ddRNAi expression vector comprises one or more ddRNAi expression cassettes. In one preferred embodiment, the ddRNAi expression vector comprises a viral derived vector and even more preferably an Adeno-Associated Virus (AAV) derived vector.

The present invention further contemplates pharmaceutical compositions comprising an RNAi agent and/or a ddRNAi expression construct as described herein together with a pharmaceutically acceptable carrier or diluent. Compositions for systemic and/or topical application are specifically contemplated.

In another aspect, the present invention contemplates genetically modified cells comprising a ddRNAi expression construct as described herein, or a genomically integrated form or part thereof. Preferably the cell is a mammalian cell, even more preferably the cell is a primate or rodent cell and most preferably the cell is a human or mouse cell. Furthermore, in yet another aspect, the present invention contemplates a multicellular structure comprising one or more genetically modified cells of the present invention. Multicellular structures include, inter alia, include a tissue, organ or complete organism.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

A list of abbreviations used herein is provided in Table 1.

TABLE 1
Abbreviations

| Abbreviation | Description |
|---|---|
| AAV | Adeno-Associated Virus |
| ddRNAi | DNA-directed RNAi |
| DHT | 5α-Dihydrotestosterone |
| HAT | Hair Associated genetic Target |
| RNAi | RNA interference |
| shRNA | Short hairpin RNA |
| siRNA | Synthetic RNA |

A summary of sequence identifiers used throughout the subject specification is provided in Table 2.

TABLE 2
Summary of sequence identifiers

| SEQ ID NO: | DESCRIPTION |
|---|---|
| 1 | DHT receptor encoding nucleotide sequence |
| 2 | 5α-reductase α-polypeptide 1 encoding nucleotide |
| | sequence |
| 3 | 5α-reductase α-polypeptide 2 encoding nucleotide |
| | sequence |

### BRIEF DESCRIPTION OF THE FIGURES

FIG. 1 is a graphical representation showing a simplified flow chart showing the steps of a method according to one embodiment of the present invention in which an RNAi expression construct is used. FIG. 1A depicts Method 100, which includes a step 200 in which an RNAi expression cassette targeting a HAT is constructed. Next, in step 300, the RNAi expression cassette is ligated into an appropriate viral delivery construct. The viral RNAi expression delivery construct is then packaged into viral particles at step 400, and the viral particles are delivered to the target cells, tissue, organ or organism at step 500. FIG. 1B shows an alternative embodiment of the method shown in FIG. 1A, where non-viral vectors are employed.

FIG. 2 is a graphical representation showing single-promoter and multiple-promoter ddRNAi expression cassettes. FIGS. 2A and 2B are simplified schematics of single-promoter RNAi expression cassettes. FIG. 2A shows an embodiment of a single RNAi expression cassette (10) comprising one [promoter-RNAi-terminator] component (shown at 20), where the ddRNAi agent is expressed initially as a shRNA. FIG. 2B shows an embodiment of a single RNAi expression cassette (10) with one [promoter-RNAi-terminator component] (shown at 20), where the sense and antisense components of the ddRNAi agent are expressed separately from different promoters. FIGS. 2C and 2D are simplified schematics of multiple-promoter RNAi expression cassettes according to embodiments of the present invention. FIG. 2C shows an embodiment of a multiple-promoter RNAi expression cassette (10) comprising three [promoter-RNAi-terminator] components (shown at 20), and FIG. 2D shows an embodiment of a multiple-promoter expression cassette (10) with five [promoter-RNAi-terminator] (shown at 20). P1, P2, P3, P4 and P5 represent promoter elements. RNAi1, RNAi2, RNAi3, RNAi4 and RNAi5 represent sequences for five different ddRNAi agents. T1, T2, T3, T4, and T5 represent termination elements.

FIGS. 3A-3D are graphical representations showing schematics of multiple ddRNAi expression cassettes or “multiple-promoter” expression cassettes. FIGS. 3A and 3B show multiple-promoter ddRNAi expression cassettes that express shRNAs. A, B and C represent three different promoter elements, and the arrows indicate the direction of transcription. Term1, Term2, and Term3 represent three different termination sequences, and shRNA-1, shRNA-2 and shRNA-3 represent three different shRNA sequences. The multiple-promoter RNAi expression cassettes in both embodiments extend from the box marked A to the Term3. FIG. 3A shows each of the three [promoter-RNAi-terminator] components (20) in the same orientation within the cassette, while FIG. 3B shows the [promoter-RNAi-terminator] components for shRNA-1 and shRNA-3 in one orientation, and the [promoter-RNAi-terminator] component for sh-RNA2 in the opposite orientation. FIGS. 3C and 3D show multiple-promoter RNAi expression constructs comprising multiple-promoter RNAi expression cassettes that express RNAi agents without a hairpin loop. In both figures, P1, P2, P3, P4, P5 and P6 represent promoter elements (with arrows indicating the direction of transcription); and T1, T2, T3, T4, T5, and T6 represent termination elements. RNAi1 sense and RNAi1 antisense (a/s) are complements, RNAi2 sense and RNAi2 a/s are complements, and RNAi3 sense and RNAi3 a/s are complements.

FIGS. 4A and 4B are graphical representations showing alternative methods for packaging the ddRNAi expression constructs of the present invention into viral particles for delivery. In FIG. 4A, a RNAi expression cassette is ligated to a viral delivery vector (step 300), and the resulting viral RNAi expression construct is used to transfect packaging cells (step 410). The packaging cells then replicate viral sequences, express viral proteins and package the viral RNAi expression constructs into infectious viral particles (step 420). The method shown in FIG. 4B utilizes cells for packaging that do not stably express viral replication and packaging genes. In this case, the RNAi expression construct is ligated to the viral delivery vector (step 300) and then co-transfected with one or more vectors that express the viral sequences necessary for replication and production of infectious viral particles (step 470). The cells replicate viral sequences, express viral proteins and package the viral RNAi expression constructs into infectious viral particles (step 420).

### DETAILED DESCRIPTION OF THE PREFERRED EMBODIMENTS

Before describing the present invention in detail, it is to be understood that unless otherwise indicated, the subject invention is not limited to specific formulations, synthesis methods, therapeutic protocols, or the like as such may vary. It is also to be understood that the terminology used herein is for the purpose of describing particular embodiments only and is not intended to be limiting.

It must be noted that, as used in the subject specification, the singular forms “a”, “an” and “the” include plural aspects unless the context already dictates otherwise. Thus, for example, reference to “a hair associated genetic target” includes a single hair associated genetic target as well as two or more hair associated genetic targets; a “a genetic construct” includes a single construct as well as two or more constructs; and so forth.

Unless defined otherwise, all technical and scientific terms used herein have the same meaning as commonly understood by one of ordinary skill in the art to which this invention belongs.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the present invention.

The present invention is directed to agents and methods for modulating the growth, development and/or maintenance of hair or hair follicles in vertebrate animals.

As used herein, the term “vertebrate animal” encompasses mammals, avian species, fish or reptiles. Preferably, the vertebrate animal is a mammal. The term “Mammals” includes humans and non-human primates, livestock animals (e.g. sheep, cow, goat, pig, donkey, horse), laboratory test animals (e.g. rat, mouse, rabbit, guinea pig, hamster), companion animals (e.g. dog, cat) or captured wild animals. Particularly preferred mammals include human and murine mammals.

The present invention provides a method for modulating the growth, development or maintenance of hair or hair follicles in an animal subject together with RNAi agents for use therewith, genetic constructs which encode RNAi agents and genetically modified cells comprising the genetic constructs. The present invention is predicated, in part, on the application of agents which facilitate gene silencing via RNAi to downregulate or silence one or more transcriptionally active genetic regions which are directly or indirectly associated with the growth, development and/or maintenance of hair or hair follicles. Such transcriptionally active regions are also referred to herein as “hair associated genetic targets” or “HATs”. RNAi-mediated silencing of one or more HATs effects modulation, including promotion or suppression, of the growth, development or maintenance of hair or hair follicles in the subject.

The term “RNA interference” or “RNAi” refers generally to a process in which a double-stranded RNA molecule changes the expression of a nucleic acid sequence with which the double-stranded or short hairpin RNA molecule shares substantial or total homology. The term or “RNAi agent” refers to an RNA sequence that elicits RNAi; and the term “ddRNAi agent” refers to an RNAi agent that is transcribed from a vector. The terms “short hairpin RNA” or “shRNA” refer to an RNA structure having a duplex region and a loop region. This term should also be understood to specifically include RNA molecules with stem-loop or panhandle secondary structures. In some embodiments of the present invention, ddRNAi agents are expressed initially as shRNAs. The terms “RNAi expression cassette” and “ddRNAi expression cassette” refer to cassettes according to embodiments of the present invention having at least one [promoter-RNAi agent-terminator] unit. The term “multiple promoter RNAi expression cassette” refers to an RNAi expression cassette comprising two or more [promoter-RNAi agent-terminator] units. The terms “RNAi expression construct” or “RNAi expression vector” refer to vectors containing at least one RNAi expression cassette.

As used herein, the terms “hair associated genetic target” or “HAT” refers to any genetic sequence or transcript thereof which is directly or indirectly associated with the growth, development and/or maintenance of hair or hair follicles in a vertebrate animal, particularly mammalian animals and most particularly in primate or rodent animals. Accordingly, a HAT may be a gene directly associated with hair growth or a transcript thereof, a nucleic acid region which encodes for a regulatory RNA, such as an efference RNA (eRNA) which is associated with hair growth or development, or the HAT may comprise a protein-encoding or regulatory RNA encoding nucleic acid sequence which itself may not be associated with hair growth or development, but the expression of which may modulate the expression of a gene or regulatory RNA which is directly associated with hair growth or development. Accordingly, the term HAT should be understood to include genetic targets which directly or indirectly modulate hair growth or development in a vertebrate animal subject.

Reference herein to “modulating the growth, development and/or maintenance of hair or hair follicles” encompasses both promoting and/or inhibiting the growth, development or maintenance of hair or hair follicles, depending on which is desired. For example, promotion or inhibition of hair growth will be determined in part by the choice of HAT which is targeted. For example, ddRNAi-mediated silencing of a HAT such as the DHT receptor-encoding gene may be used to promote the process of hair growth. However, suppression of a wild type gene at the hairless locus may effect hair growth suppression. Accordingly, as would be evident to one of skill in the art, the methods of the present invention may be adapted to either promote or inhibit the process of hair growth, depending on, inter alia, the particular HAT which is targeted for silencing.

In one embodiment, the HAT is a gene which encodes an “androgenic steroid hormone interacting protein”, which refers to any protein which interacts with one or more androgenic steroids directly, or a protein which indirectly interacts with a pathway which is signalled by one or more androgenic steroids and which mediate the process of hair growth or loss in animal subjects.

The androgenic steroid hormone interacting protein comprises the androgen receptor or 5α-dihydrotestosterone (DHT) receptor. DHT is a metabolic derivative of the major circulating androgenic steroid hormone testosterone, arising generally from the intracellular conversion of testosterone within androgen-responsive cells by the enzyme steroid 5α-reductase.

In a particularly preferred embodiment, the DHT receptor or homolog or ortholog thereof is encoded by a nucleotide sequence comprising the sequence set forth in SEQ ID NO:1 or a nucleotide sequence with at least 70% identity thereto.

In another preferred embodiment, the androgenic steroid hormone interacting protein is steroid 5α-reductase. Steroid 5α-reductase comprises two forms, α-polypeptide 1 and α-polypeptide 2. Reference herein to “steroid 5α-reductase” is taken as reference to either or both forms.

In further preferred embodiments, the steroid 5α-reductase α-polypeptide 1 is encoded by the nucleotide sequence set forth in SEQ ID NO:2 or a nucleotide sequence with at least 70% identity thereto. In yet another preferred embodiment the steroid 5α-reductase α-polypeptide 2 is encoded by the nucleotide sequence set forth in SEQ ID NO:3 or a nucleotide sequence with at least 70% identity thereto.

Reference herein to “at least 70% identity” includes percentage identities of 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100%.

Although the genes encoding the DHT receptor and steroid 5α-reductase represent preferred HATs, the present invention contemplates the modulation of other HATs using ddRNAi. Other exemplary HATs which may be targeted to modulate hair or hair follicle growth, development or maintenance in a vertebrate animal subject include, but are in no way limited to the genetic sequences and transcripts thereof presented in Table 3.

TABLE 3
Exemplary HAT sequences which may be targeted using ddRNAi

| | Entrez |
|---|---|
| HAT | Gene ID No. |
| Hr (Hairless)¹ | 55806 |
| Lah/Dsg 4 (Desmoglein 4)² | 147409 |
| Shh (Sonic hedgehog)³ | 6469 |
| Vegf⁴ | 7422 |
| Cd34 (Cd34 antigen)⁵ | 947 |
| S100a4⁵ | 6275 |
| Idb2 (Idb2 helix-loop-helix antagonist)⁵ | 9079 |
| Idb4⁵ | 30579 |
| Peg3 (Paternally expressed gene 3)⁵ | 5178 |
| Fzd2 (Frizzled 2)⁵ | 2535 |
| Dkk3 (Dickkopf homolog 3)⁵ | 27122 |
| Sfrp1 (Secreted Frizzled Related Protein 1)⁵ | 6422 |
| Dab2 (Disabled homolog 2)⁵ | 1601 |
| Cktsf1b1 (Gremlin, cysteine knot superfamily 1, BMP | 26585 |
| antagonist 1)⁵ | |
| Fgfr1 (Fibroblast growth factor receptor 1)⁵ | 2260 |
| Fgf1 (Fibroblast growth factor 1)⁵ | 2246 |
| Gpr49 (G-protein-coupled receptor 49)⁵ | 8549 |
| Igfbp5 (Insulin-linke growth factor binding protein 5)⁵ | 3488 |
| Myoc (Trabecular meshwork induced glucocorticoid | 4653 |
| protein)⁵ | |
| Itm2a (Integral membrane protein 2A)⁵ | 9452 |
| Eps8 (Epidermal growth factor receptor pathway | 2059 |
| substrate 8) | |
| Fyn (Fyn proto-oncogene)⁵ | 2534 |
| Col6a1 (Procollagen, type IV, alpha 1)⁵ | 1291 |
| Tnc (Tenascin C)⁵ | 3371 |
| Krt2-6a (Keratin complex 2, basic, gene 6a)⁵ | 16687 |
| Potassium channel subfamily K member 2⁵ | 3776 |
| Skd3 (Suppressor of K+ transport defect 3)⁵ | 81570 |
| Clic4 (Chloride intracellular channel 4)⁵ | 25932 |
| Col18a1 (Endostatin, alpha 1 (XVIII) collagen)⁵ | 80781 |
| Gna 14 (Guanine nucleotide binding protein)⁵ | 9630 |
| Ly6 (Lymphocyte antigen 6 complex)⁵ | 17062 |
| Bmp4 (Bone morphogenetic protein 4)⁵ | 652 |
| II1r2 (Interleukin 1 receptor, type II)⁵ | 7850 |
| Wnt3a (Wingless-related MMTV integration site 3A)⁵ | 89780 |
| II12rb2 (Interleukin 12 receptor, beta 2)⁵ | 3595 |
| Wnt10a (Wingless-related MMTV integration site 10a)⁵ | 80326 |
| Ifngr2 (Interferon-gamma receptor precursor)⁵ | 3460 |
| Fgfbp1 (Fibroblast growth factor binding protein 1)⁵ | 9982 |
| Klf5 (Kruppel-like factor 5)⁵ | 688 |
| Gata3 (GATA binding protein 3)⁵ | 2625 |
| Mki67 (antigen identified by monoclonal antibody Ki-67)⁵ | 4288 |
| Cks2 (CDC28 protein kinase regulatory subunit 2)⁵ | 1164 |
| Ccng2 (Cyclin G2), Prc1 (Protein regulator of cytokinesis | 901 |
| 1)⁵ | |

¹Ahmad et al., Science 279: 720-724, 1998
²Kljuic et al., Cell 113: 249-260, 2003
³Sato et al., J. Clin. Invest. 104: 855-864, 1999
⁴U.S. Pat. No. 6,773,881
⁵Morris et al., Nature Biotechnology 22(4): 411-417, 2004

The present invention is predicated in part on the use of RNAi agents to silence the expression of one or more HATs, which in turn either promotes or inhibits hair growth, development or maintenance in a vertebrate animal subject. The term “silencing of expression” in this context includes regulating the amount of functional RNA transcript derived from the HAT. Regulating the amount of functional RNA transcript may occur by facilitating transcript degradation or facilitating formation of nucleic acid based molecules which inhibit translation. In either case, the RNAi agents promote or facilitate post-transcriptional gene silencing. As used herein “functional RNA transcript” refers to an RNA transcript which is able to perform its usual function. For example, in the case of the HAT being a protein encoding gene, a “functional RNA transcript” would be a translatable mRNA. However, in the case of a HAT which encodes a non-translated regulatory RNA, a “functional RNA transcript” would be an RNA transcript capable of effecting regulation of another genetic sequence.

RNAi is generally optimised by identical sequences between the target and the RNAi agent. The RNA interference phenomenon can be observed with less than 100% homology, but the complementary regions must be sufficiently homologous to each other to form the specific double stranded regions. The precise structural rules to achieve a double-stranded region effective to result in RNA interference have not been fully identified, but approximately 70% identity is generally sufficient. Accordingly, in some embodiments of the invention, the homology between the RNAi agent and the HAT is at least 70% nucleotide sequence identity, and may be at least 75% nucleotide sequence identity. Preferably, homology is at least 80% nucleotide sequence identity, and is at least 85% or even 90% nucleotide sequence identity. More preferably, sequence homology between the target sequence and the sense strand of the RNAi is at least 90%, 91%, 92%, 93%, 94%, 95%, 96%, 97%, 98%, 99% or 100% nucleotide sequence identity.

Another consideration is that base-pairing in RNA is subtly different from DNA in that G will pair with U, although not as strongly as it does with C, in RNA duplexes. Moreover, for RNAi efficacy, it is more important that the antisense strand be homologous to the target sequence. In some circumstances, it is known that 17 out of 21 nucleotides is sufficient to initiate RNAi, but in other circumstances, identity of 19 or 20 nucleotides out of 21 is required. It is believed, at a general level, that greater homology is required in the central part of a double stranded region than at its ends. Some predetermined degree of lack of perfect homology may be designed into a particular construct so as to reduce its RNAi activity which would result in a partial silencing or repression of the target gene's product, in circumstances in which only a degree of silencing was sought. In such a case, it is envisaged that only one or two bases of the antisense sequence would be changed. On the other hand, the other, sense strand is more tolerant of mutations. It is believed this is due to the antisense strand being the one that is catalytically active. Thus, less identity between the sense strand and the transcript of a region of a target gene will not necessarily reduce RNAi activity, particularly where the antisense strand perfectly hybridizes with that transcript. Mutations in the sense strand (such that it is not identical to the transcript of the region of the target gene) may be useful to assist sequencing of hairpin constructs and potentially for other purposes, such as modulating dicer processing of a hairpin transcript or other aspects of the RNAi pathway.

The terms “hybridizing” and “annealing” (and grammatical equivalents) are used interchangeably in this specification in respect of nucleotide sequences and refer to nucleotide sequences that are capable of forming Watson-Crick base pairs due to their complementarity. The person skilled in the art would understand that non-Watson-Crick base-pairing is also possible, especially in the context of RNA sequences. For example a so-called “wobble pair” can form between guanosine and uracil residues in RNA. “Complementary” is used herein in its usual way to indicate Watson-Crick base pairing, and “non-complementary” is used to mean non-Watson-Crick base pairing, even though such non-complementary sequences may form wobble pairs or other interactions. However, in the context of the present invention, reference to “non-pairing” sequences relates specifically to sequences between which Watson-Crick base pairs do not form. Accordingly, embodiments of spacing or bubble sequences according to the present invention are described and illustrated herein as non-pairing sequences, regardless of whether non-Watson-Crick base pairing could theoretically or does in practice occur.

Terms used to describe sequence relationships between two or more polynucleotides include “reference sequence”, “comparison window”, “sequence similarity”, “sequence identity”, “percentage of sequence similarity”, “percentage of sequence identity”, “substantially similar” and “substantial identity”. A “reference sequence” is at least 10 but frequently 15 to 25 and often greater than 25 or above, such as 30 monomer units, inclusive of nucleotides, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically at least about 10 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (eg. GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. A detailed discussion of sequence analysis can be found in Unit 19.3 of Ausubel et al. For example, “percentage of sequence identity”, may be calculated by comparing two optimally aligned sequences over the window of comparison, determining the number of positions at which the identical nucleic acid base (e.g. A, T, C, G, I) occurs in both sequences to yield the number of matched positions, dividing the number of matched positions by the total number of positions in the window of comparison (ie., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

The RNAi agents of the present invention preferably comprise short, double-stranded, or partially double-stranded (eg. panhandle, stem-loop and hairpin) RNAs that are not toxic in normal mammalian cells. There is no particular limitation in the length of the RNAi agents of the present invention as long as they do not show cellular toxicity. RNAi agents can be, for example, 15 to 49 bp in length, preferably 15 to 35 bp in length, and are more preferably 19 to 29 bp in length. The double-stranded RNA portions of RNAis may be completely homologous, or may contain non-paired portions due to sequence mismatch (the corresponding nucleotides on each strand are not complementary), bulge (lack of a corresponding complementary nucleotide on one strand), and the like. Such non-paired portions can be tolerated to the extent that they do not significantly interfere with RNAi duplex formation or efficacy.

An entire HAT-transcript may be targeted by the RNAi agent or shorter segments or portions may be targeted.

The term “transcript” is used here to include any functional RNA transcribed from a transcriptionally active HAT. By “part” means at least about 10 contiguous nucleotide but less than the entire nucleotide sequence. Examples of parts of a transcript include from about 10 to about 500 nucleotides, from about 12 to about 200 nucleotides, from about 15 to about 100 nucleotides and from about 15 to about 50 nucleotides. Particularly preferred target lengths of nucleotides in the transcripts are from about 18 to 30 and even more preferably lengths are from about 20 to 24 such as 20 or 21 or 22 or 23 or 24 nucleotides.

The RNAi agents and/or ddRNAi expression cassettes contemplated herein comprise a “targeting sequence” which includes a nucleotide sequence which is at least 70% identical to at least part of the nucleotide sequence of a HAT or a complement thereof. The targeting sequence may comprise any length of nucleotides that is able to induce a gene silencing effect or otherwise reduce the level of translatable transcript. Preferably, the targeting sequence is from 10 to 500 nucleotides in length, more preferably 10 to 50 nucleotides in length, even more preferably 15, 16, 17, 18, 19, 20, 21, 22, 23, 24, 25, 26, 27, 28, 29 or 30 nucleotides in length and most preferably 20, 21, 22, 23 or 24 nucleotides in length.

Preferably, the RNAi agents are directed to regions or parts of the HAT which are conserved. Methods of alignment of sequences for comparison and RNAi sequence selection are well known in the art. The determination of percent identity between two or more sequences can be accomplished using a mathematical algorithm. Preferred, non-limiting examples of such mathematical algorithms are the algorithm of Myers and Miller (Comput. Appl. Biosci. 4: 11-17, 1988); the search-for-similarity-method of Pearson and Lipman (Proc Natl Acad Sci USA. 85(8): 2444-8, 1988); and that of Karlin and Altschul (Proc. Natl. Acad. Sci. USA 87: 2264-2268, 1990). Preferably, computer implementations of these mathematical algorithms are utilized. Such implementations include, but are not limited to: CLUSTAL in the PC/Gene program (available from Intelligenetics, Mountain View, Calif.); the ALIGN program (Version 2.0), GAP, BESTFIT, BLAST, FASTA, Megalign (using Jotun Hein, Martinez, Needleman-Wunsch algorithms), DNAStar Lasergene (see www.dnastar.com) and TFASTA in the Wisconsin Genetics Software Package, Version 8 (available from Genetics Computer Group (GCG), 575 Science Drive, Madison, Wis., USA). Alignments using these programs can be performed using the default parameters or parameters selected by the operator. The CLUSTAL program is well described by Higgins. The ALIGN program is based on the algorithm of Myers and Miller supra; and the BLAST programs are based on the algorithm of Karlin and Altschul, supra. Software for performing BLAST analyses is publicly available through the National Center for Biotechnology Information (http://www.ncbi.nim.nih.gov/).

In one preferred embodiment the targeting sequence comprises a 20, 21, 22, 23 or 24 mer sense nucleotide sequences from the DHT receptor gene as set forth in SEQ ID NO:1 or a nucleotide sequence with at least 70% identity thereto. A targeting sequence derived from the corresponding cDNA molecule encoding an entire DHT receptor may also be employed.

In another preferred embodiment, a similar approach can be taken for steroid 5α-reductase genes 1 and 2. Accordingly in another preferred embodiment the ddRNAi targeting sequence comprises a 20, 21, 22, 23 or 24 mer sense nucleotide sequences from the steroid 5α-reductase polypeptide 1 gene as set forth in SEQ ID NO:2 or a nucleotide sequence with at least 70% identity thereto. In yet another preferred embodiment the ddRNAi targeting sequence comprises a 20, 21, 22, 23 or 24 mer sense nucleotide sequences from the steroid 5α-reductase polypeptide 2 gene as set forth in SEQ ID NO:3 or a nucleotide sequence with at least 70% identity thereto.

In yet further preferred embodiments, the targeting sequence comprises a 20, 21, 22, 23 or 24 mer sense sequence derived from any of the hairless (hr) locus (Ahmad et al., 1998, supra), the lanceolate hair (lah) locus, Dsg4 (Desmoglein 4) (Kljuic et al, 2003, supra), Shh (Sonic hedgehog) (Sato et al., 1999, supra), Vegf (U.S. Pat. No. 6,773,881), Cd34 (Cd34 antigen), S100, Ibd2 (Ibd2 helix-loop-helix antagonist), Ibd4, Peg3 (Paternally expressed gene 3), Fzd2 (Frizzled 2), Dkk3 (Dickkopf homolog 3), Sfrp1 (Secreted Frizzled Related Protein 1), Dab2 (Disabled homolog 2), Cktsflb1 (Gremlin, cysteine knot superfamily 1, BMP antagonist 1), Fgfr1 (Fibroblast growth factor receptor 1), Fgt1 (Fibroblast growth factor 1), Gpr49 (G-protein-coupled receptor 49), Igfbp5 (Insulin-linke growth factor binding protein 5), Myoc (Trabecular meshwork induced glucocorticoid protein), Itm2a (Integral membrane protein 2A), Eps8 (Epidermal growth factor receptor pathway substrate 8), Fyn (Fyn proto-oncogene), Col6a1 (Procollagen, type IV, alpha 1), Tnc (Tenascin C), Krt2-6a (Keratin complex 2, basic, gene 6a), Potassium channel subfamily K encoding sequences, Skd3 (Suppressor of K+ transport defect 3), Clic4 (Chloride intracellular channel 4), Col18a1 (Endostatin, alpha 1 (XVIII) collagen), Gna14 (Guanine nucleotide binding protein), Ly6 (Lymphocyte antigen 6 complex), Bmp4 (Bone morphogenetic protein 4), II1r2 (Interleukin 1 receptor, type II), Wnt3a (Wingless-related MMTV integration site 3A), II12rb2 (Interleukin 12 receptor, beta 2), Wnt10a (Wingless-related MMTV integration site 10a), Ifngr2 (Interferon-gamma receptor precursor), Fgfbp1 (Fibroblast growth factor binding protein 1), Klf5 (Kruppel-like factor 5), Gata3 (GATA binding protein 3), Retinoic acid stimulated basic helix-loop-helix protein encoding sequences, Mki67 (antigen identified by monoclonal antibody Ki-67), Cks2 (CDC28 protein kinase regulatory subunit 2), Ccng2 (Cyclin G2), Prc1 (Protein regulator of cytokinesis 1) (Morris et al., 2004, supra).

The termini of an RNAi agent may be blunt or cohesive (overhanging) as long as the RNAi agent effectively silences the target gene. The cohesive (overhanging) end structure is not limited only to a 3′ overhang, but a 5′ overhanging structure may be included as long as the resulting RNAi agent is capable of inducing the RNAi effect. In addition, the number of overhanging nucleotides may be any number as long as the resulting RNAi agent is capable of inducing the RNAi effect. For example, if present, the overhang may consist of 1 to 8 nucleotides, preferably it consists of 2 to 4 nucleotides.

Preferred RNAi agents include siRNAs (synthetic RNAs) or DNA-directed RNAs (ddRNAs).

“siRNAs” or short interfering RNAs may be manufactured by methods known in the art such as by typical oligonucleotide synthesis, and often will incorporate chemical modifications to increase half life and/or efficacy of the siRNA agent, and/or to allow for a more robust delivery formulation. Many modifications of oligonucleotides are known in the art. For example, U.S. Pat. No. 6,620,805 discloses an oligonucleotide that is combined with a macrocycle having a net positive charge such as a porphyrin; U.S. Pat. No. 6,673,611 discloses various formulas; US Patent Application Publication Nos. 2004/0171570, 2004/0171032, and 2004/0171031 disclose oligomers that include a modification comprising a polycyclic sugar surrogate; such as a cyclobutyl nucleoside, cyclopentyl nucleoside, proline nucleoside, cyclohexene nucleoside, hexose nucleoside or a cyclohexane nucleoside; and oligomers that include a non-phosphorous-containing internucleoside linkage; US Patent Application Publication No. 2004/0171579 discloses a modified oligonucleotide where the modification is a 2′ substituent group on a sugar moiety that is not H or OH; US Patent Application Publication No. 2004/0171030 discloses a modified base for binding to a cytosine, uracil, or thymine base in the opposite strand comprising a boronated C and U or T modified binding base having a boron-containing substituent selected from the group consisting of —BH₂CN, —BH₃, and —BH₂COOR, wherein R is C1 to C18 alkyl; US Patent Application Publication No. 2004/0161844 discloses oligonucleotides having phosphoramidate internucleoside linkages such as a 3′aminophosphoramidate, aminoalkylphosphoramidate, or aminoalkylphosphorthioamidate internucleoside linkage; US Patent Application Publication No. 2004/0161844 discloses yet other modified sugar and/or backbone modifications, where in some embodiments, the modification is a peptide nucleic acid, a peptide nucleic acid mimic, a morpholino nucleic acid, hexose sugar with an amide linkage, cyclohexenyl nucleic acid (CeNA), or an acyclic backbone moiety; US Patent Application Publication No. 2004/0161777 discloses oligonucleotides with a 3′ terminal cap group; US Patent Application Publication No. 2004/0147470 discloses oligomeric compounds that include one or more cross-linkages that improve nuclease resistance or modify or enhance the pharmacokinetic and phamacodynamic properties of the oligomeric compound where such cross-linkages comprise a disulfide, amide, amine, oxime, oxyamine, oxyimine, morpholino, thioether, urea, thiourea, or sulfonamide moiety; US Patent Application Publication No. 2004/0147023 discloses a gapmer comprising two terminal RNA segments having nucleotides of a first type and an internal RNA segment having nucleotides of a second type where nucleotides of said first type independently include at least one sugar substituent where the sugar substituent comprises a halogen, amino, trifluoroalkyl, trifluoroalkoxy, azido, aminooxy, alkyl, alkenyl, alkynyl, O—, S—, or N(R*)-alkyl; O—, S—, or N(R*)-alkenyl; O—, S— or N(R*)—alkynyl; O—, S— or N-aryl, O—, S—, or N(R*)-aralkyl group; where the alkyl, alkenyl, alkynyl, aryl or aralkyl may be a substituted or unsubstituted alkyl, alkenyl, alkynyl, aralkyl; and where, if substituted, the substitution is an alkoxy, thioalkoxy, phthalimido, halogen, amino, keto, carboxyl, nitro, nitroso, cyano, trifluoromethyl, trifluoromethoxy, imidazole, azido, hydrazino, aminooxy, isocyanato, sulfoxide, sulfone, disulfide, silyl, heterocycle, or carbocycle group, or an intercalator, reporter group, conjugate, polyamine, polyamide, polyalkylene glycol, or a polyether of the formula (—O-alkyl)ₘ, where m is 1 to about 10; and R* is hydrogen, or a protecting group; or US Patent Application Publication No. 2004/0147022 disclosing an oligonucleotide with a modified sugar and/or backbone modification, such as a 2′-OCH₃ substituent group on a sugar moiety.

Accordingly, in one aspect, the present invention contemplates a method for modulating the growth, development and/or maintenance of hair or hair follicles in a subject, said method comprising administering to said subject an siRNA comprising a nucleotide sequence which is at least 70% identical to at least part of a nucleotide sequence comprising a HAT or a derivative, ortholog or homolog thereof and which delays, represses or otherwise reduces the expression of the HAT in said subject.

“ddRNAs” include RNAi agents transcribed or otherwise derived from constructs and vectors which comprise one or more ddRNAi expression cassettes. Such constructs are also referred to herein as “ddRNAi expression vectors” or “ddRNAi expression constructs”. Administration of such genetic agents to animal cells is proposed to transiently or permanently reduce the amount of functional RNA transcript associated with the HAT and thereby reduce inhibit the functional expression of the HAT.

As used herein, the terms “vector”, “construct”, “ddRNAi expression vector” or “ddRNAi expression construct” may include replicons such as plasmids, phage, viral constructs, cosmids, Bacterial Artificial Chromosomes (BACs), Yeast Artificial Chromosomes (YACs) Human Artificial Chromosomes (HACs) and the like into which one or more ddRNAi expression cassettes may be or are ligated.

The genetic construct in effect comprises a ddRNAi expression cassette. As used herein, the term “ddRNAi expression cassette” refers to a nucleic acid sequence which is able to effect transcription to produce an RNAi agent. Preferably, this includes a nucleic acid molecules being single or double stranded, partially double stranded, stem-loop and/or a panhandle type molecule. Typically, a ddRNAi expression cassette comprises a promoter operably linked to a ddRNAi targeting sequence which in turn is operably linked to a terminator.

Accordingly, another aspect of the present invention contemplates a method for modulating the growth, development and/or maintenance of hair or hair follicles in a subject, said method comprising administering to said subject a genetic construct comprising at least one ddRNAi expression cassette which encodes an RNA molecule comprising a nucleotide sequence which is at least 70% identical to at least part of a nucleotide sequence comprising a HAT or a derivative, ortholog or homolog thereof and which delays, represses or otherwise reduces the expression of the HAT in said subject.

Preferably, the ddRNAi expression cassettes contemplated herein comprise a ddRNAi targeting sequence under the operable control of one or more regulatory sequences, including, inter alia, a promoter sequence which is operable in a target cell tissue or organ.

Reference herein to a “promoter” or “promoter sequence” is to be taken in its broadest context and includes a DNA regulatory region capable of binding RNA polymerase in a cell and initiating transcription of a polynucleotide or polypeptide coding sequence such as messenger RNA, ribosomal RNAs, small nuclear of nucleolar RNAs or any kind of RNA transcribed by any class of any RNA polymerase. “Promoters” contemplated herein may also include the transcriptional regulatory sequences of a classical genomic gene, including the TATA box which is required for accurate transcription initiation in eukaryotic cells, with or without a CCAAT box sequence and additional regulatory elements (i.e. upstream activating sequences, enhancers and silencers).

A promoter is usually, but not necessarily, positioned upstream or 5′, of the sequence which it regulates. Furthermore, the regulatory elements comprising a promoter are usually positioned within 2 kb of the start site of transcription of the sequence to be regulated.

In the present context, the term “promoter” is also used to describe a synthetic or fusion molecule or derivative which confers, activates or enhances expression of an isolated nucleic acid molecule in a mammalian cell. Another or the same promoter may also be required to function in plant, animal, insect, fungal, yeast or bacterial cells. Preferred promoters may contain additional copies of one or more specific regulatory elements to further enhance expression of a structural gene, which in turn regulates and/or alters the spatial expression and/or temporal expression of the gene. For example, regulatory elements which confer inducibility on the expression of the structural gene may be placed adjacent to a heterologous promoter sequence driving expression of a nucleic acid molecule.

Placing a sequence under the regulatory control of a promoter sequence means positioning said molecule such that expression is controlled by the promoter sequence. Promoters are generally positioned 5′ (upstream) to the genes that they control. In the construction of heterologous promoter/structural gene combinations, it is generally preferred to position the promoter at a distance from the gene transcription start site that is approximately the same as the distance between that promoter and the gene it controls in its natural setting, i.e. the gene from which the promoter is derived. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e. the genes from which it is derived. Again, as is known in the art, some variation in this distance can also occur.

The promoter may regulate the expression of a sequence constitutively, or differentially with respect to the cell, tissue or organ in which expression occurs, or with respect to the developmental stage at which expression occurs, or in response to stimuli such as physiological stresses, regulatory proteins, hormones, pathogens or metal ions, amongst others.

Promoters useful in some embodiments of the present invention may be tissue-specific or cell-specific. The term “tissue specific” as it applies to a promoter refers to a promoter that is capable of directing selective expression of a nucleotide sequence of interest to a specific type of tissue (eg. follicular skin tissue), in the relative absence of expression of the same nucleotide sequence of interest in a different type of tissue (e.g., brain). The term “cell-specific” as applied to a promoter refers to a promoter which is capable of directing selective expression of a nucleotide sequence of interest in a specific type of cell in the relative absence of expression of the same nucleotide sequence of interest in a different type of cell within the same tissue (see, eg., Higashibata et al., J. Bone Miner. Res. 19(1): 78-88, 2004; Hoggatt et al., Circ. Res. 91(12): 1151-59, 2002; Sohal et al., Circ. Res. 89(1): 20-25, 2001; and Zhang et al., Genome Res. 14(1): 79-89, 2004). The term “cell-specific” when applied to a promoter also means a promoter capable of promoting selective expression of a nucleotide sequence of interest in a region within a single tissue. Alternatively, promoters may be constitutive or regulatable. Additionally, promoters may be modified so as to possess different specificities.

The term “constitutive” when made in reference to a promoter means that the promoter is capable of directing transcription of an operably linked nucleic acid sequence in the absence of a specific stimulus (eg., heat shock, chemicals, light, etc.). Typically, constitutive promoters are capable of directing expression of a coding sequence in substantially any cell and any tissue. The promoters used to transcribe the RNAi agents preferably are constitutive promoters, such as the promoters for ubiquitin, CMV, β-actin, histone H4, EF-1□ or pgk genes controlled by RNA polymerase II, or promoter elements controlled by RNA polymerase I. In other embodiments, a Pol II promoter such as CMV, SV40, U1, β-actin or a hybrid Pol II promoter is employed. In other embodiments, promoter elements controlled by RNA polymerase III are used, such as the U6 promoters (U6-1, U6-8, U6-9, e.g.), H1 promoter, 7SL promoter, the human Y promoters (hY1, hY3, hY4 (see Maraia et al., Nucleic Acids Res 22(15): 3045-52, 1994) and hY5 (see Maraia et al., Nucleic Acids Res 24(18): 3552-59, 1994), the human MRP-7-2 promoter, Adenovirus VA1 promoter, human tRNA promoters, the 5s ribosomal RNA promoters, as well as functional hybrids and combinations of any of these promoters.

Alternatively, in some embodiments it may be optimal to select promoters that allow for inducible expression of the RNAi agent. A number of systems for inducible expression using such promoters are known in the art, including but not limited to the tetracycline responsive system and the lac operator-repressor system (see WO 03/022052 A1; and US 2002/0162126 A1), the ecdyson regulated system, or promoters regulated by glucocorticoids, progestins, estrogen, RU-486, steroids, thyroid hormones, cyclic AMP, cytokines, the calciferol family of regulators, or the metallothionein promoter (regulated by inorganic metals).

One or more enhancers also may be present in the viral multiple-promoter RNAi expression construct to increase expression of the gene of interest. Enhancers appropriate for use in embodiments of the present invention include the Apo E HCR enhancer, the CMV enhancer that has been described recently (see, Xia et al, Nucleic Acids Res 31-17, 2003), and other enhancers known to those skilled in the art.

Preferably, the promoter is capable of regulating expression of a nucleic acid molecule in a mammalian cell, at least during the period of time over which the target gene is expressed therein and more preferably also immediately preceding the commencement of detectable expression of the target gene in said cell. Promoters may be constitutive, inducible or developmentally regulated.

In the present context, the terms “in operable connection with” or “operably under the control” or similar such as “operably linked to” shall be taken to indicate that expression of the structural gene is under the control of the promoter sequence with which it is spatially connected in a cell.

In some embodiments, promoters of variable strength may be employed within a single ddRNAi expression cassette or between different cassettes in a ddRNAi expression vector which comprises multiple ddRNAi expression cassettes. For example, use of two or more strong promoters (such as a Pol III-type promoter) may tax the cell, by, e.g., depleting the pool of available nucleotides or other cellular components needed for transcription. In addition or alternatively, use of several strong promoters may cause a toxic level of expression of RNAi agents in the cell. Thus, in some embodiments one or more of the promoters in the multiple-promoter RNAi expression cassette may be weaker than other promoters in the cassette, or all promoters in the cassette may express RNAi agents at less than a maximum rate. Promoters also may or may not be modified using molecular techniques, or otherwise, e.g., through regulation elements, to attain weaker levels of transcription.

Accordingly, another aspect of the present invention contemplates a method for modulating the growth, development and/or maintenance of hair or hair follicles in a subject, said method comprising administering to said subject a genetic construct which comprises a ddRNAi expression cassette which encodes an RNA molecule comprising a nucleotide sequence which is at least 70% identical to at least part of a nucleotide sequence comprising a HAT or a derivative, ortholog or homolog thereof and which delays, represses or otherwise reduces the expression of the HAT in said subject wherein the sequence which encodes the RNA molecule is operably connected to a promoter.

As stated, the ddRNAi agent coding regions of ddRNAi expression cassette are operatively linked to terminator elements. In one embodiment, the terminators comprise stretches of four or more thymidine residues. In embodiments where multiple promoter cassettes are used, the terminator elements used all may be different and are matched to the promoter elements from the gene from which the terminator is derived. Such terminators include the SV40 poly A, the Ad VA1 gene, the 5S ribosomal RNA gene, and the terminators for human t-RNAs. In addition, promoters and terminators may be mixed and matched, as is commonly done with RNA pol II promoters and terminators.

Accordingly, yet another aspect of the present invention contemplates a method for modulating the growth, development and/or maintenance of hair or hair follicles in a subject, said method comprising administering to said subject a genetic construct comprising at least one ddRNAi expression cassette which encodes an RNA molecule comprising a nucleotide sequence which is at least 70% identical to at least part of a nucleotide sequence comprising a HAT or a derivative, ortholog or homolog thereof and which delays, represses or otherwise reduces the expression of the HAT in said subject wherein the sequence which encodes the RNA molecule is operably connected to a promoter and a terminator region.

In one preferred embodiment, the ddRNAi expression cassette comprises a nucleic acid molecule comprising the general structure (I): 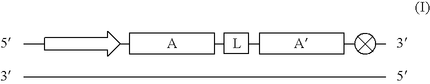
wherein: [symbol]
represents a promoter sequence; [symbol]
represents a ddRNAi targeting sequence comprising at least 10 nucleotides, wherein said sequence is at least 70% identical to a HAT sequence or part thereof; [symbol]
represents a sequence of 10 to 30 nucleotides wherein at least 10 contiguous nucleotides of A′ comprise a reverse complement of the nucleotide sequence represented by A; [symbol]
represents a “loop” encoding structure comprising a sequence of 5 to 20 non-self-complementary nucleotides; and [symbol]
represents a terminator sequence.

Accordingly, another aspect of the present invention contemplates a method for modulating the growth, development and/or maintenance of hair or hair follicles in a subject, said method comprising administering to said subject a genetic construct which comprises at least one ddRNAi expression cassette which encodes an RNA molecule comprising a nucleotide sequence which is at least 70% identical to at least part of a nucleotide sequence comprising a HAT or a derivative, ortholog or homolog thereof and which delays, represses or otherwise reduces the expression of the HAT in said subject, wherein at least one of said ddRNAi expression cassettes comprises the general structure (I).

The ddRNAi agent generated by the expression of the ddRNAi expression cassette represented by general structure (I) comprises a stem-loop structured precursor (shRNA) in which the ends of the double-stranded RNA are connected by a single-stranded, linker RNA. The length of the single-stranded loop portion of the shRNA may be 5 to 20 bp in length, and is preferably 5 to 9 bp in length. Accordingly, in a preferred embodiment, L in general structure (I) comprises 5, 6, 7, 8 or 9 non-self-complementary nucleotides.

In another preferred embodiment, the ddRNAi expression cassette comprises a nucleic acid molecule of the general structure (II): 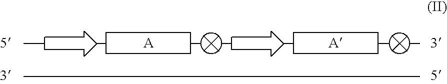
wherein: [symbol]
represents a promoter sequence; [symbol]
represents a ddRNAi targeting sequence comprising at least 10 nucleotides, wherein said sequence is at least 70% identical to a HAT sequence or part thereof; [symbol]
represents a sequence of 10 to 30 nucleotides wherein at least 10 contiguous nucleotides of A′ comprise a reverse complement of the nucleotide sequence represented by A; and [symbol]
represents a terminator sequence.

Accordingly, another aspect of the present invention contemplates a method for modulating the growth, development and/or maintenance of hair or hair follicles in a subject, said method comprising administering to said subject a genetic construct which comprises at least one ddRNAi expression cassette which encodes an RNA molecule comprising a nucleotide sequence which is at least 70% identical to at least part of a nucleotide sequence comprising a HAT or a derivative, ortholog or homolog thereof and which delays, represses or otherwise reduces the expression of the HAT in said subject, wherein at least one of said ddRNAi expression cassettes comprises the general structure (II).

In yet another embodiment, the ddRNAi expression cassette comprises a nucleic acid molecule of the general structure (III): 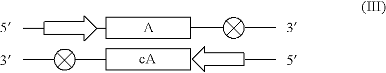
wherein: [symbol]
represents a promoter sequence; [symbol]
represents a ddRNAi targeting sequence comprising at least 10 nucleotides, wherein said sequence is at least 70% identical to a HAT sequence or part thereof; [symbol]
represents a nucleic acid sequence complementary to A; and [symbol] represents a terminator sequence.

Accordingly, another aspect of the present invention contemplates a method for modulating the growth, development and/or maintenance of hair or hair follicles in a subject, said method comprising administering to said subject a genetic construct which comprises at least one ddRNAi expression cassette which encodes an RNA molecule comprising a nucleotide sequence which is at least 70% identical to at least part of a nucleotide sequence comprising a HAT or a derivative, ortholog or homolog thereof and which delays, represses or otherwise reduces the expression of the HAT in said subject, wherein at least one of said ddRNAi expression cassettes comprises the general structure (III).

In yet another preferred embodiment, the ddRNAi expression cassette comprises a nucleic acid molecule of the general structure (IV): 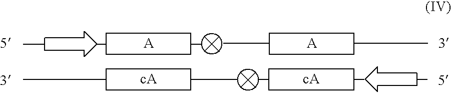
wherein: [symbol]
represents a promoter sequence; [symbol]
represents a ddRNAi targeting sequence comprising at least 10 nucleotides, wherein said sequence is at least 70% identical to a HAT sequence or part thereof; [symbol]
represents a nucleic acid sequence complementary to A; and [symbol]
represents a terminator sequence.

Accordingly, another aspect of the present invention contemplates a method for modulating the growth, development and/or maintenance of hair or hair follicles in a subject, said method comprising administering to said subject a genetic construct which comprises at least one ddRNAi expression cassette which encodes an RNA molecule comprising a nucleotide sequence which is at least 70% identical to at least part of a nucleotide sequence comprising a HAT or a derivative, ortholog or homolog thereof and which delays, represses or otherwise reduces the expression of the HAT in said subject, wherein at least one of said ddRNAi expression cassettes comprises the general structure (IV).

Although the ddRNAi expression cassettes represented by general structures (I), (II), (III) and (IV) represent preferred embodiments of the invention, the present invention is in no way limited to these particular general structures. As would be evident to one of skill in the art, the above structures may be modified while retaining functionality. For example, the elements of the cassettes may be separated by one or more nucleotide residues. Furthermore, elements which are present on complementary strands, such as the terminator and promoter elements shown in structures (III) and (IV) may overlap or may be discreet. For example, the terminator elements shown in structure (III) may occur within the complementary strand of the promoter element or may be upstream or downstream of this region. Other modifications which would be evident to one of skill in the art and which do not materially effect the functioning of the cassette in encoding a dsRNA stucture may also be made and such modified cassettes are within the scope of the present invention.

In addition, the ddRNAi expression cassettes may be configured where multiple cloning sites and/or unique restriction sites are located strategically, such that the promoter, ddRNAi agent-encoding sequence and terminator elements are easily removed or replaced. The RNAi expression cassettes may be assembled from smaller oligonucleotide components using strategically located restriction sites and/or complementary sticky ends. The base vector for one approach according to embodiments of the present invention consists of plasmids with a multilinker in which all sites are unique (though this is not an absolute requirement). Sequentially, each promoter is inserted between its designated unique sites resulting in a base cassette with one or more promoters, all of which can have variable orientation. Sequentially, again, annealed primer pairs are inserted into the unique sites downstream of each of the individual promoters, resulting in a single-, double- or multiple-expression cassette construct. The insert can be moved into, e.g. an AAV backbone using two unique enzyme sites (the same or different ones) that flank the single-, double- or multiple-expression cassette insert.

Accordingly, in another aspect, the present invention contemplates a ddRNAi expression cassette as described herein, wherein said ddRNAi expression cassette, once expressed in a host cell, effects transcription of an RNAi agent which effects RNAi-mediated silencing of one or more HATs. In preferred embodiments of the invention, the ddRNAi comprises the general structure of any one of general structures (I), (II), (III) or (IV) or derivatives or variants thereof.

One or more ddRNAi expression cassette may be ligated into any convenient vector or construct for delivery, expression and/or replication in a target cell. A vector or construct comprising one or more ddRNAi expression cassettes is referred to herein as a “ddRNAi expression vector” or “ddRNAi expression construct”.

The constructs into which the RNAi expression cassette is inserted and used for high efficiency transduction and expression of the ddRNAi agents in various cell types may be, inter alia, derived from viruses and are compatible with viral delivery; alternatively, non-viral delivery method may be used. Generation of the construct can be accomplished using any suitable genetic engineering techniques well known in the art, including without limitation, the standard techniques of PCR, oligonucleotide synthesis, restriction endonuclease digestion, ligation, transformation, plasmid purification, and DNA sequencing. If the construct is a viral construct, the construct preferably comprises, for example, sequences necessary to package the RNAi expression construct into viral particles and/or sequences that allow integration of the RNAi expression construct into the target cell genome. The viral construct also may contain genes that allow for replication and propagation of virus, though in other embodiments such genes will be supplied in trans. Additionally, the viral construct may contain genes or genetic sequences from the genome of any known organism incorporated in native form or modified. For example, a preferred viral construct may comprise sequences useful for replication of the construct in bacteria.

The genetic constructs contemplated herein may also comprise more than one ddRNAi expression cassette as described herein. For example, a single ddRNAi expression vector may comprise 1, 2, 3, 4, 5, 6, 7, 8, 9 or 10 ddRNAi cassettes in a single vector. Each cassette may be of the same general structure or may be different and each may comprise the same or different ddRNAi targeting sequences. Furthermore, each ddRNAi expression cassette may be in “forward” or “reverse” orientation.

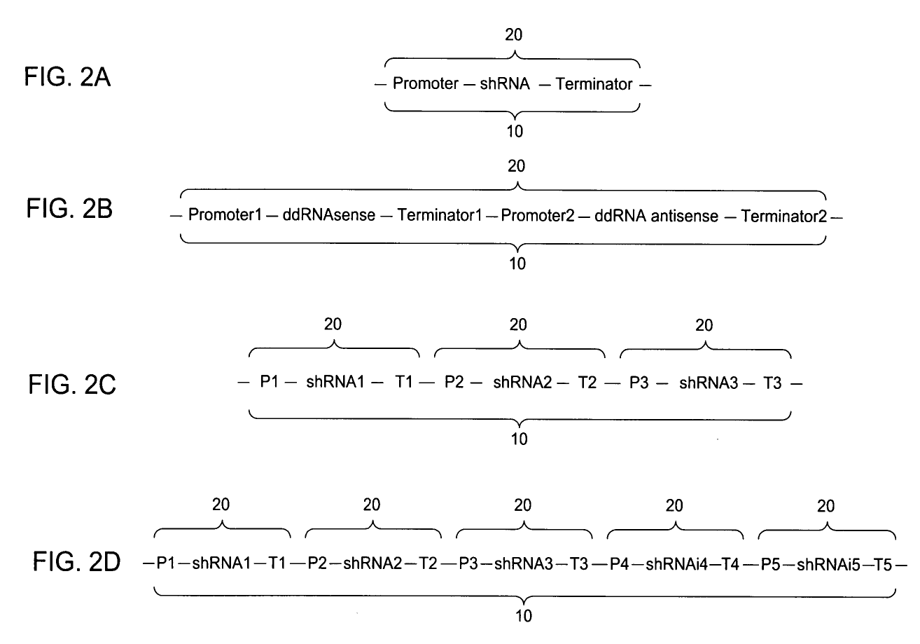

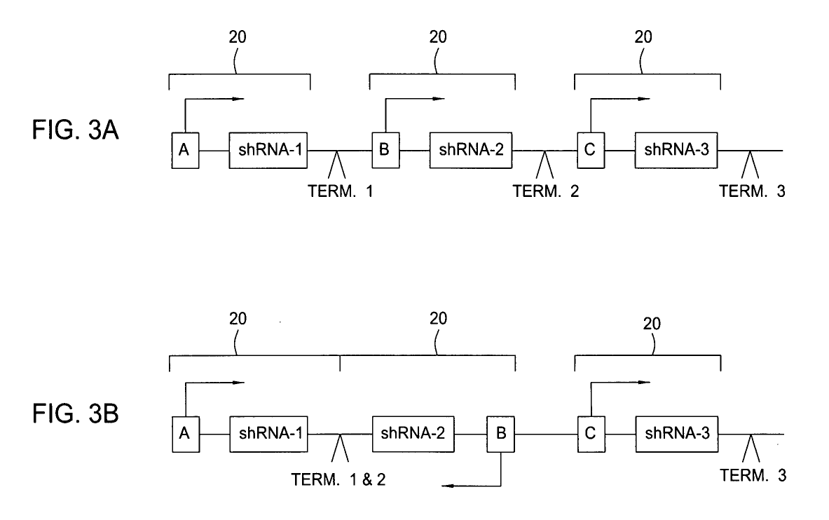

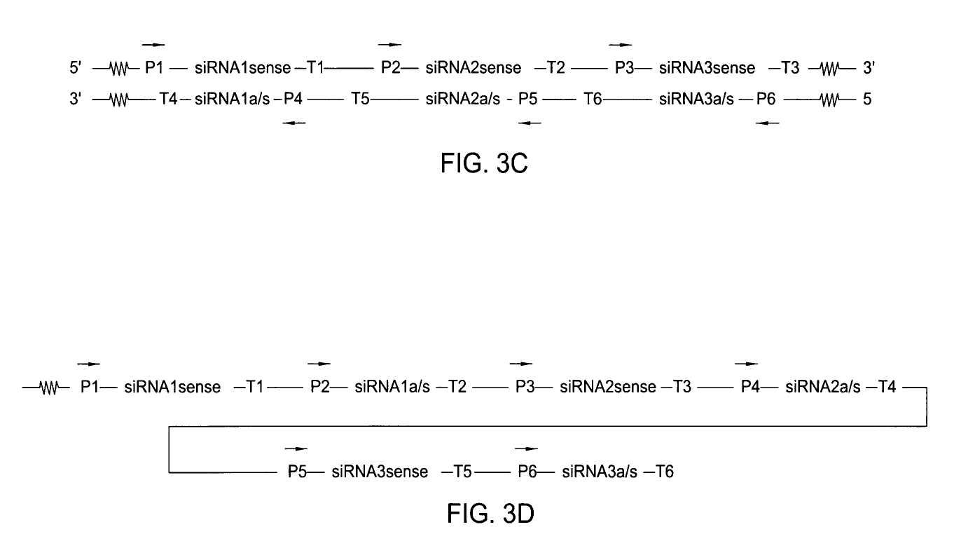

In one embodiment, a ddRNAi expression construct or vector which comprises more than one ddRNAi expression cassette is a “multiple promoter” construct. Exemplary schematic structures of such constructs are set out in FIGS. 2B, C and D and FIGS. 3A, B, C and D.

The optimum number of ddRNAi expression cassettes which may be involved in the genetic constructs of the present invention will vary considerably, depending upon the length of each of the ddRNAi targeting gene sequences, their orientation and degree of identity to each other. For example, those skilled in the art will be aware of the inherent instability of palindromic nucleotide sequences in vivo and the difficulties associated with constructing long synthetic genes comprising inverted repeated nucleotide sequences, because of the tendency for such sequences to form hairpin loops and to recombine in vivo. Notwithstanding such difficulties, the optimum number of ddRNAi targeting gene sequences to be included in the genetic constructs of the present invention may be determined empirically by those skilled in the art, without any undue experimentation and by following standard procedures such as the construction of the synthetic gene of the invention using recombinase-deficient cell lines, reducing the number of repeated sequences to a level which eliminates or minimizes recombination events and by keeping the total length of the multiple ddRNAi expression cassette sequence to an acceptable limit, preferably no more than 5-10 kb, more preferably no more than 2-5 kb and even more preferably no more than 0.5-2.0 kb in length.

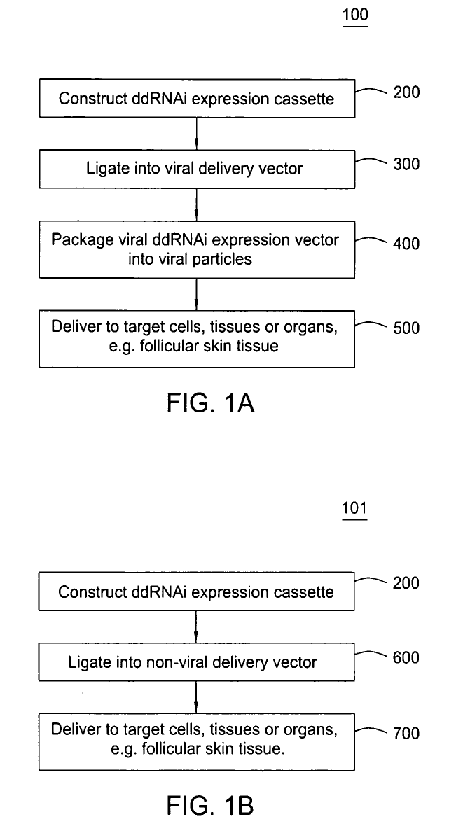

FIG. 1A is a simplified flow chart showing the steps of a method according to one embodiment of the present invention in which an RNAi expression construct according to the present invention may be used. Method 100 includes a step 200 in which an RNAi expression cassette targeting a HAT is constructed. Next, in step 300, the RNAi expression cassette is ligated into an appropriate viral delivery construct. The viral RNAi expression delivery construct is then packaged into viral particles at step 400, and the viral particles are delivered to the target cells, tissue, organ or organism at step 500. Details for each of these steps and the components involved are presented infra. FIG. 1B shows an alternative embodiment of the method shown in FIG. 1A, where non-viral vectors are employed.

Viral-based RNAi expression constructs according to the present invention can be generated synthetically or enzymatically by a number of different protocols known to those of skill in the art and purified using standard recombinant DNA techniques as described in, for example, Sambrook et al., Molecular Cloning: A Laboratory Manual, 2nd Ed., Cold Spring Harbor Press, Cold Spring Harbor, N.Y. (1989), and under regulations described in, e.g., United States Dept. of HHS, National Institute of Health (NIH) Guidelines for Recombinant DNA Research.

FIGS. 2A and 2B are simplified schematics of single-promoter RNAi expression cassettes according to embodiments of the present invention. FIG. 2A shows an embodiment of a single RNAi expression cassette (10) comprising one [promoter-RNAi-terminator] component (shown at 20), where the ddRNAi agent is expressed initially as a shRNA. FIG. 2B shows an embodiment of a single RNAi expression cassette (10) with one [promoter-RNAi-terminator] component (shown at 20), where the sense and antisense components of the ddRNAi agent are expressed separately from different promoters.

FIGS. 2C and 2D are simplified schematics of multiple-promoter RNAi expression cassettes according to embodiments of the present invention. FIG. 2C shows an embodiment of a multiple-promoter RNAi expression cassette (10) comprising three [promoter-RNAi-terminator] components (shown at 20), and FIG. 2D shows an embodiment of a multiple-promoter expression cassette (10) with five [promoter-RNAi-terminator] components (shown at 20). P1, P2, P3, P4 and P5 represent promoter elements. RNAi1, RNAi2, RNAi3, RNAi4 and RNAi5 represent sequences for five different ddRNAi agents. T1, T2, T3, T4, and T5 represent termination elements. The multiple-promoter RNAi expression cassettes according to the present invention may contain two or more [promoter-RNAi-terminator] components where the number of [promoter-RNAi-terminator] components included in any multiple-promoter RNAi expression cassette is limited by, e.g., packaging size of the delivery system chosen (for example, some viruses, such as AAV, have relatively strict size limitations); cell toxicity, and maximum effectiveness (i.e. when, for example, expression of four ddRNAi agents is as effective therapeutically as the expression of ten ddRNAi agents).

When employing a multiple promoter RNAi expression cassette, the two or more ddRNAi agents in the [promoter-RNAi-terminator] components comprising a cassette all have different sequences; that is RNAi1, RNAi2, RNAi3, RNAi4 and RNAi5 are all different from one another. However, the promoter elements in any cassette may be the same (that is, e.g., the sequence of two or more of P1, P2, P3, P4 and P5 may be the same); all the promoters within any cassette may be different from one another; or there may be a combination of promoter elements represented only once and promoter elements represented two times or more within any cassette. Similarly, the termination elements in any cassette may be the same (that is, e.g., the sequence of two or more of T1, T2, T3, T4 and T5 may be the same, such as contiguous stretches of 4 or more T residues); all the termination elements within any cassette may be different from one another; or there may be a combination of termination elements represented only once and termination elements represented two times or more within any cassette. Preferably, the promoter elements and termination elements in each [promoter-RNAi-terminator] component comprising any cassette are all different to decrease the likelihood of DNA recombination events between components and/or cassettes. Further, in a preferred embodiment, the promoter element and termination element used in each [promoter-RNAi-terminator] component are matched to each other; that is, the promoter and terminator elements are taken from the same gene in which they occur naturally.

FIGS. 3A and 3B show multiple-promoter RNAi expression constructs comprising alternative embodiments of multiple-promoter RNAi expression cassettes that express short shRNAs. shRNAs are short duplexes where the sense and antisense strands are linked by a hairpin loop. Once expressed, shRNAs are processed into RNAi agents. A, B and C represent three different promoter elements, and the arrows indicate the direction of transcription. Term1, Term2, and Term3 represent three different termination sequences, and shRNA-1, shRNA-2 and shRNA-3 represent three different shRNA sequences. The multiple-promoter RNAi expression cassettes in both embodiments extend from the box marked A to the Term3. FIG. 3A shows each of the three [promoter-RNAi-terminator] components (20) in the same orientation within the cassette, while FIG. 3B shows the [promoter-RNAi-terminator] components for shRNA-1 and shRNA-3 in one orientation, and the [promoter-RNAi-terminator] component for sh-RNA2 in the opposite orientation (i.e., transcription takes place on both strands of the cassette). Other variations may be used as well.

FIGS. 3C and 3D show multiple-promoter RNAi expression constructs comprising alternative embodiments of multiple-promoter RNAi expression cassettes that express RNAi agents without a hairpin loop. In both figures, P1, P2, P3, P4, P5 and P6 represent promoter elements (with arrows indicating the direction of transcription); and T1, T2, T3, T4, T5, and T6 represent termination elements. Also in both figures, RNAi1 sense and RNAi1 antisense (a/s) are complements, RNAi2 sense and RNAi2 a/s are complements, and RNAi3 sense and RNAi3 a/s are complements.

In the embodiment shown in FIG. 3C, all three RNAi sense sequences are transcribed from one strand (via P1, P2 and P3), while the three RNAi a/s sequences are transcribed from the complementary strand (via P4, P5, P6). In this particular embodiment, the termination element of RNAi1 a/s (T4) falls between promoter P1 and the RNAi 1 sense sequence; while the termination element of RNAi1 sense (T1) falls between the RNAi 1 a/s sequence and its promoter, P4. This motif is repeated such that if the top strand shown in FIG. 3C is designated the (+) strand and the bottom strand is designated the (−) strand, the elements encountered moving from left to right would be P1(+), T4(−), RNAi1 (sense and a/s), T1(+), P4(−), P2(+), T5(−), RNAi2 (sense and a/s), T2(+), P5(−), P3(+), T6(−), RNAi3 (sense and a/s), T3(+), and P6(−).

In an alternative embodiment shown in FIG. 3D, all RNAi sense and antisense sequences are transcribed from the same strand. One skilled in the art appreciates that any of the embodiments of the multiple-promoter RNAi expression cassettes shown in FIGS. 3A through 3D may be used for certain applications, as well as combinations or variations thereof.

The construct also may contain additional genetic elements. The types of elements that may be included in the construct are not limited in any way and may be chosen by one of skill in the art. For example, additional genetic elements may include a reporter gene, such as one or more genes for a fluorescent marker protein such as GFP or RFP; an easily assayed enzyme such as beta-galactosidase, luciferase, beta-glucuronidase, chloramphenical acetyl transferase or secreted embryonic alkaline phosphatase; or proteins for which immunoassays are readily available such as hormones or cytokines. Other genetic elements that may find use in embodiments of the present invention include those coding for proteins which confer a selective growth advantage on cells such as adenosine deaminase, aminoglycodic phosphotransferase, dihydrofolate reductase, hygromycin-B-phosphotransferase, drug resistance, or those genes coding for proteins that provide a biosynthetic capability missing from an auxotroph. If a reporter gene is included along with the RNAi expression cassette, an internal ribosomal entry site (IRES) sequence can be included. Preferably, the additional genetic elements are operably linked with and controlled by an independent promoter/enhancer. In addition, a suitable origin of replication for propagation of the construct in bacteria may be employed. The sequence of the origin of replication generally is separated from the ddRNAi agent and other genetic sequences that are to be expressed in the target cell, tissue and/or organ. Such origins of replication are known in the art and include the pUC, ColE1, 2-micron or SV40 origins of replication.

The genetic constructs described herein or parts thereof may also be adapted for integration into the genome of a cell in which it is expressed. Those skilled in the art will be aware that, in order to achieve integration of a genetic sequence or genetic construct into the genome of a host cell, certain additional genetic sequences may be required.

In one embodiment, the effect of the genetic construct is to reduce functional expression of the HAT while not substantially reducing the level of transcription of the HAT. Alternatively or in addition to, the genetic construct including synthetic gene does not result in a substantial reduction in steady state levels of total RNA.

Accordingly, in another aspect, the present invention contemplates a ddRNAi expression construct wherein said ddRNAi expression construct comprises one or more ddRNAi expression cassettes as defined herein.

The RNAi agents of the present invention, result in or otherwise facilitate an altered capacity for translation of a target transcript for translation into an expression product. Although the expression product is generally a protein, the present invention further contemplates expression products in the form of transcribed non-coding RNAs, eRNAs or introns spliced out of a transcript which are involved in genetic regulation.

Reference to “altered capacity” preferably includes a reduction in the level of translation such as from about 10% to about 100% and more preferably from about 20% to about 90% relative to a cell which is not genetically modified. In a particularly preferred embodiment, the gene corresponding to the target endogenous sequence is substantially not translated into a proteinaceous product. Conveniently, an altered capacity of translation is determined by any change of phenotype wherein the phenotype, in a non-genetically modified cell, is facilitated by the expression of a gene encoding a HAT. Any cell carrying a genetic agent of the present invention is said to be “genetically modified”. The genetic modification may be permanent or transient. A transient genetic modification occurs, for example, when a cell takes up a genetic agent and permits the generation of transcript. Alternatively, the genetic agent directly reduces the level of translation such as by the administration of an antisense oligonucleotide or larger nucleic acid molecule. In either case, the molecule facilitates gene silencing mechanisms which, as the cells divide, may be removed. Permanent gene silencing is more likely to occur when the genetic agent integrates into a cell's genome and the agent is passed onto daughter cells.

Although the present invention is particularly directed to animal or human hair follicle-forming skin cells, the scope of the invention extends to any vertebrate animal cell.

Preferably the vertebrate animal cells are derived from mammals, avian species, fish or reptiles. Preferably, the vertebrate animal cells are derived from mammals. Mammalian cells may be from a human, primate, livestock animal (e.g. sheep, cow, goat, pig, donkey, horse), laboratory test animal (e.g. rat, mouse, rabbit, guinea pig, hamster), companion animal (e.g. dog, cat) or captured wild animal. Particularly preferred mammalian cells are from human and murine animals. Furthermore, the present invention provides a genetically modified animal useful as an animal model. Such animal models are readily generated by grafting, for example, hair follicle-producing cells onto the skin of an animal such as a mouse. Nude mice are particularly useful in this regard as recipients of human hair follicle-producing cells. Such animals are useful test models for the genetic agents of the present invention.

Standard methods may be used to administer the RNAi agents or ddRNAi expression constructs to a cell, tissue or organ for the purposes of modulating the expression of the target gene. Useful methods of administration include liposome-mediated transfection or transformation, transformation of cells with attenuated virus particles or bacterial cells, cell mating, transformation or transfection procedures known to those skilled in the art or described by Ausubel et al. (1992). For example, a nucleic acid molecule may be introduced as naked DNA or RNA, optionally encapsulated in a liposome, in a virus particle as attenuated virus or associated with a virus coat or a transport protein or inert carrier such as gold or as a recombinant viral vector or bacterial vector or as a genetic construct, amongst others.

In one embodiment, a viral delivery system based on any appropriate virus may be used to deliver the ddRNAi expression constructs of the present invention. In addition, hybrid viral systems may be of use. The choice of viral delivery system will depend on various parameters, such as efficiency of delivery into follicular skin tissue or other target tissues, transduction efficiency of the system, pathogenicity, immunological and toxicity concerns, and the like. It is clear that there is no single viral system that is suitable for all applications. When selecting a viral delivery system to use in the present invention, it is important to choose a system where ddRNAi expression construct-containing viral particles are preferably: 1) reproducibly and stably propagated; 2) able to be purified to high titers; and 3) able to mediate targeted delivery (delivery of the multiple-promoter RNAi expression construct to the target tissue (eg. follicular skin tissue) without widespread dissemination).

In general, the five most commonly used classes of viral systems used in gene therapy can be categorized into two groups according to whether their genomes integrate into host cellular chromatin (oncoretroviruses and lentiviruses) or persist in the cell nucleus predominantly as extrachromosomal episomes (adeno-associated virus, adenoviruses and herpesviruses).

For example, in one embodiment of the present invention, viruses from the Parvoviridae family are utilized. The Parvoviridae is a family of small single-stranded, non-enveloped DNA viruses with genomes approximately 5000 nucleotides long. Included among the family members is adeno-associated virus (AAV), a dependent parvovirus that by definition requires co-infection with another virus (typically an adenovirus or herpesvirus) to initiate and sustain a productive infectious cycle. In the absence of such a helper virus, AAV is still competent to infect or transduce a target cell by receptor-mediated binding and internalization, penetrating the nucleus in both non-dividing and dividing cells.

Once in the nucleus, the virus uncoats and the transgene is expressed from a number of different forms—the most persistent of which are circular monomers. AAV will integrate into the genome of 1-5% of cells that are stably transduced (Nakai et al., J. Virol. 76: 11343-349, 2002). Expression of the transgene can be exceptionally stable and in one study with AAV delivery of Factor IX, a dog model continues to express therapeutic levels of the protein 4.5 years after a single direct infusion with the virus. Because progeny virus is not produced from AAV infection in the absence of helper virus, the extent of transduction is restricted only to the initial cells that are infected with the virus. It is this feature which makes AAV a preferred gene therapy vector for the present invention. Furthermore, unlike retrovirus, adenovirus, and herpes simplex virus, AAV appears to lack human pathogenicity and toxicity (Kay et al., Nature 424: 251, 2003 and Thomas et al., Nature Reviews, Genetics 4: 346-58, 2003).

Typically, the genome of AAV contains only two genes. The “rep” gene codes for at least four separate proteins utilized in DNA replication. The “cap” gene product is spliced differentially to generate the three proteins that comprise the capsid of the virus. When packaging the genome into nascent virus, only the Inverted Terminal Repeats (ITRs) are obligate sequences; rep and cap can be deleted from the genome and be replaced with heterologous sequences of choice. However, in order produce the proteins needed to replicate and package the AAV-based heterologous construct into nascent virion, the rep and cap proteins must be provided in trans. The helper functions normally provided by co-infection with the helper virus, such as adenovirus or herpesvirus mentioned above, also can be provided in trans in the form of one or more DNA expression plasmids. Since the genome normally encodes only two genes it is not surprising that, as a delivery vehicle, AAV is limited by a packaging capacity of 4.5 single stranded kilobases (kb). However, although this size restriction may limit the genes that can be delivered for replacement gene therapies, it does not adversely affect the packaging and expression of shorter sequences such as RNAi.

The utility of AAV for RNAi applications was demonstrated in experiments where AAV was used to deliver shRNA in vitro to inhibit p53 and Caspase 8 expression (Tomar et al., Oncogene 22: 5712-15, 2003). Following cloning of the appropriate sequences into a gutted AAV-2 vector, infectious AAV virions were generated in HEK293 cells and used to infect HeLa S3 cells. A dose-dependent decrease of endogenous Caspase 8 and p53 levels was demonstrated. Boden et al. also used AAV to deliver shRNA in vitro to inhibit HIV replication in tissue culture systems (Boden et al., J. Virol. 77(21): 115231-35, 2003) as assessed by p24 production in the spent media.

However, technical hurdles must be addressed when using AAV as a vehicle for RNAi expression constructs. For example, various percentages of the human population may possess neutralizing antibodies against certain AAV serotypes. However, since there are several AAV serotypes, some of which the percentage of individuals harboring neutralizing antibodies is vastly reduced, other serotypes can be used or pseudo-typing may be employed. There are at least eight different serotypes that have been characterized, with dozens of others which have been isolated but have been less well described. Another limitation is that as a result of a possible immune response to AAV, AAV-based therapy may only be administered once; however, use of alternate, non-human derived serotypes may allow for repeat administrations. Administration route, serotype, and composition of the delivered genome all influence tissue specificity.

Another limitation in using unmodified AAV systems with the RNAi expression constructs is that transduction can be inefficient. Stable transduction in vivo may be limited to 5-10% of cells. However, different methods are known in the art to boost stable transduction levels. One approach is utilizing pseudotyping, where AAV-2 genomes are packaged using cap proteins derived from other serotypes. For example, by substituting the AAV-5 cap gene for its AAV-2 counterpart, Mingozzi et al. increased stable transduction to approximately 15% of hepatocytes (Mingozzi et al., J. Virol. 76(20): 10497-502, 2002). Thomas et al., transduced over 30% of mouse hepatocytes in vivo using the AAV8 capsid gene (Thomas et al., J. Virol. in press). Grimm et al. (Blood. 2003-02-0495) exhaustively pseudotyped AAV-2 with AAV-1, AAV-3B, AAV-4, AAV-5, and AAV-6 for tissue culture studies. The highest levels of transgene expression were induced by virion which had been pseudotyped with AAV-6; producing nearly 2000% higher transgene expression than AAV-2. Thus, the present invention contemplates use of a pseudotyped AAV virus to achieve high transduction levels, with a corresponding increase in the expression of the RNAi multiple-promoter expression constructs.

Another viral delivery system useful with the RNAi expression constructs of the present invention is a system based on viruses from the family Retroviridae. Retroviruses comprise single-stranded RNA animal viruses that are characterized by two unique features. First, the genome of a retrovirus is diploid, consisting of two copies of the RNA. Second, this RNA is transcribed by the virion-associated enzyme reverse transcriptase into double-stranded DNA. This double-stranded DNA or provirus can then integrate into the host genome and be passed from parent cell to progeny cells as a stably-integrated component of the host genome.

In some embodiments, lentiviruses are the preferred members of the retrovirus family for use in the present invention. Lentivirus vectors are often pseudotyped with vesicular stomatitis virus glycoprotein (VSV-G), and have been derived from the human immunodeficiency virus (HIV), the etiologic agent of the human acquired immunodeficiency syndrome (AIDS); visan-maedi, which causes encephalitis (visna) or pneumonia in sheep; equine infectious anemia virus (EIAV), which causes autoimmune hemolytic anemia and encephalopathy in horses; feline immunodeficiency virus (FIV), which causes immune deficiency in cats; bovine immunodeficiency virus (BIV) which causes lymphadenopathy and lymphocytosis in cattle; and simian immunodeficiency virus (SIV), which causes immune deficiency and encephalopathy in non-human primates. Vectors that are based on HIV generally retain <5% of the parental genome, and <25% of the genome is incorporated into packaging constructs, which minimizes the possibility of the generation of reverting replication-competent HIV. Biosafety has been further increased by the development of self-inactivating vectors that contain deletions of the regulatory elements in the downstream long-terminal-repeat sequence, eliminating transcription of the packaging signal that is required for vector mobilization.

Reverse transcription of the retroviral RNA genome occurs in the cytoplasm. Unlike C-type retroviruses, the lentiviral cDNA complexed with other viral factors—known as the pre-initiation complex—is able to translocate across the nuclear membrane and transduce non-dividing cells. A structural feature of the viral cDNA—a DNA flap—seems to contribute to efficient nuclear import. This flap is dependent on the integrity of a central polypurine tract (cPPT) that is located in the viral polymerase gene, so most lentiviral-derived vectors retain this sequence. Lentiviruses have broad tropism, low inflammatory potential, and result in an integrated vector. The main limitations are that integration might induce oncogenesis in some applications. The main advantage to the use of lentiviral vectors is that gene transfer is persistent in most tissues or cell types.

A lentiviral-based construct used to express the ddRNAi agents preferably comprises sequences from the 5′ and 3′ LTRs of a lentivirus. More preferably the viral construct comprises an inactivated or self-inactivating 3′ LTR from a lentivirus. The 3′ LTR may be made self-inactivating by any method known in the art. In a preferred embodiment, the U3 element of the 3′ LTR contains a deletion of its enhancer sequence, preferably the TATA box, Sp1 and NF-kappa B sites. As a result of the self-inactivating 3′ LTR, the provirus that is integrated into the host cell genome will comprise an inactivated 5′ LTR. The LTR sequences may be LTR sequences from any lentivirus from any species. The lentiviral-based construct also may incorporate sequences for MMLV or MSCV, RSV or mammalian genes. In addition, the U3 sequence from the lentiviral 5′ LTR may be replaced with a promoter sequence in the viral construct. This may increase the titer of virus recovered from the packaging cell line. An enhancer sequence may also be included.

Other viral or non-viral systems known to those skilled in the art may be used to deliver the RNAi expression cassettes of the present invention to target tissues including follicular skin tissue, including but not limited to gene-deleted adenovirus-transposon vectors that stably maintain virus-encoded transgenes in vivo through integration into host cells (see Yant et al., Nature Biotech. 20: 999-1004, 2002); systems derived from Sindbis virus or Semliki forest virus (see Perri et al, J. Virol. 74(20): 9802-07, 2002); systems derived from Newcastle disease virus or Sendai virus; or mini-circle DNA vectors devoid of bacterial DNA sequences (see Chen et al., Molecular Therapy 8(3): 495-500, 2003).

In addition, hybrid viral systems may be used to combine useful properties of two or more viral systems. For example, the site-specific integration machinery of wild-type AAV may be coupled with the efficient internalization and nuclear targeting properties of adenovirus. AAV in the presence of adenovirus or herpesvirus undergoes a productive replication cycle; however, in the absence of helper functions, the AAV genome integrates into a specific site on chromosome 19. Integration of the AAV genome requires expression of the AAV rep protein. As conventional rAAV vectors are deleted for all viral genes including rep, they are not able to specifically integrate into chromosome 19. However, this feature may be exploited in an appropriate hybrid system. In addition, non-viral genetic elements may be used to achieve desired properties in a viral delivery system, such as genetic elements that allow for site-specific recombination.

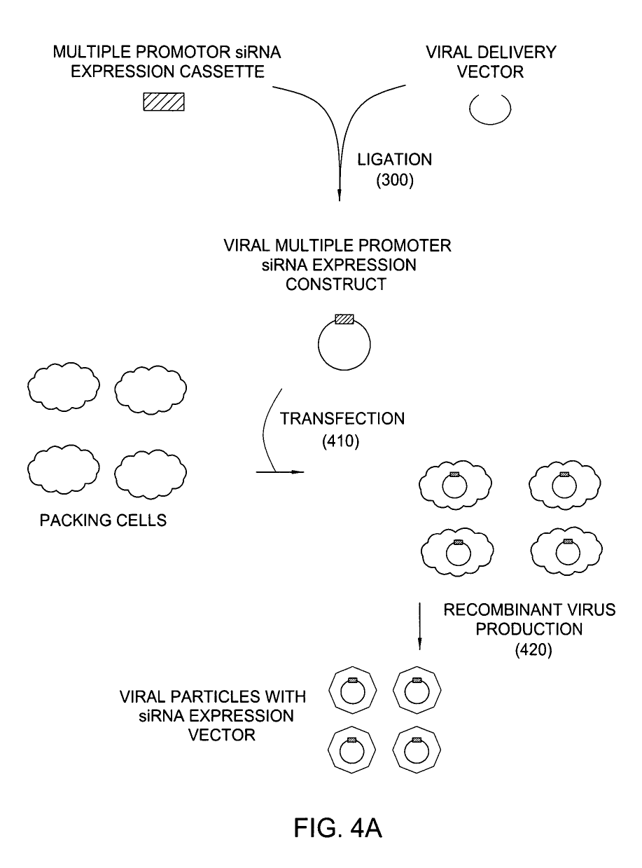

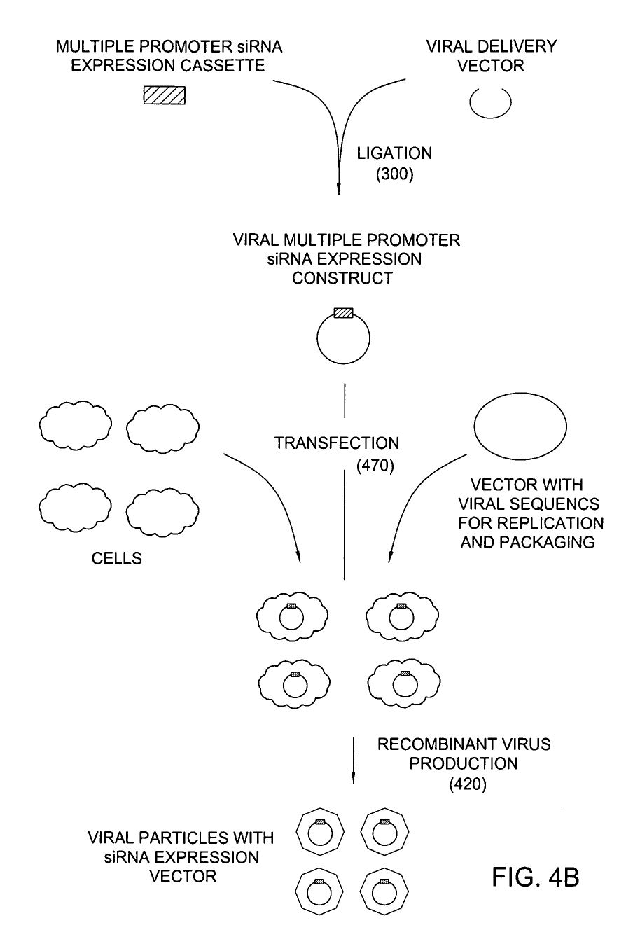

In step 400 of FIG. 1, the RNAi expression construct is packaged into viral particles. Any method known in the art may be used to produce infectious viral particles whose genome comprises a copy of the viral RNAi expression construct. FIGS. 4A and 4B show alternative methods for packaging the RNAi expression constructs of the present invention into viral particles for delivery. The method in FIG. 4A utilizes packaging cells that stably express in trans the viral proteins that are required for the incorporation of the viral RNAi expression construct into viral particles, as well as other sequences necessary or preferred for a particular viral delivery system (for example, sequences needed for replication, structural proteins and viral assembly) and either viral-derived or artificial ligands for tissue entry. In FIG. 4A, a RNAi expression cassette is ligated to a viral delivery vector (step 300), and the resulting viral RNAi expression construct is used to transfect packaging cells (step 410). The packaging cells then replicate viral sequences, express viral proteins and package the viral RNAi expression constructs into infectious viral particles (step 420). The packaging cell line may be any cell line that is capable of expressing viral proteins, including but not limited to 293, HeLa, A549, PerC6, D17, MDCK, BHK, bing cherry, phoenix, Cf2Th, or any other line known to or developed by those skilled in the art. One packaging cell line is described, for example, in U.S. Pat. No. 6,218,181.

Alternatively, a cell line that does not stably express necessary viral proteins may be co-transfected with two or more constructs to achieve efficient production of functional particles. One of the constructs comprises the viral RNAi expression construct, and the other plasmid(s) comprises nucleic acids encoding the proteins necessary to allow the cells to produce functional virus (replication and packaging construct) as well as other helper functions. The method shown in FIG. 4B utilizes cells for packaging that do not stably express viral replication and packaging genes. In this case, the RNAi expression construct is ligated to the viral delivery vector (step 300) and then co-transfected with one or more vectors that express the viral sequences necessary for replication and production of infectious viral particles (step 430). The cells replicate viral sequences, express viral proteins and package the viral RNAi expression constructs into infectious viral particles (step 420).

The packaging cell line or replication and packaging construct may not express envelope gene products. In these embodiments, the gene encoding the envelope gene can be provided on a separate construct that is co-transfected with the viral RNAi expression construct. As the envelope protein is responsible, in part, for the host range of the viral particles, the viruses may be pseudotyped. As described supra, a “pseudotyped” virus is a viral particle having an envelope protein that is from a virus other than the virus from which the genome is derived. One with skill in the art can choose an appropriate pseudotype for the viral delivery system used and cell to be targeted. In addition to conferring a specific host range, a chosen pseudotype may permit the virus to be concentrated to a very high titer. Viruses alternatively can be pseudotyped with ecotropic envelope proteins that limit infection to a specific species (e.g., ecotropic envelopes allow infection of, e.g., murine cells only, where amphotropic envelopes allow infection of, e.g., both human and murine cells.) In addition, genetically-modified ligands can be used for cell-specific targeting, such as the asialoglycoprotein for hepatocytes, or transferrin for receptor-mediated binding.

After production in a packaging cell line, the viral particles containing the ddRNAi expression cassettes are purified and quantified (titered). Purification strategies include density gradient centrifugation, or, preferably, column chromatographic methods.

The RNAi agents and/or ddRNAi expression constructs contemplated herein may be introduced to follicle-forming skin cells by topical application although the present invention also contemplates systemic administration. Topical application may be conveniently achieved with a “gene gun” or other physical means. Alternatively, the entities may be suspended within a cream or lotion or wax or other liquid solution such that topical application of the cream or lotion or wax or liquid solution results in the introduction of the entities into follicle-forming skin cells where they are capable of initiating a cellular process that abolishes or down-regulates functional RNA transcribed from a HAT.

Depending on site of application, the effect of the application is to increase the extent and/or rate of growth of scalp hair and reduce the extent and/or rate of growth of hair on other parts of the body.

“Administration” means also include injection and oral ingestion (eg. in medicated food material), amongst others, whether or not incorporated into a composition or medicament having other components. The subject nucleic acid molecules may also be delivered by a live delivery system such as using a bacterial expression system optimised for their expression in bacteria which can be incorporated into gut flora. Alternatively, a viral expression system can be employed, for example using viruses such as adenovirus, adeno-associated virus, lentiviruses and the like, as described supra. In this regard, one form of viral expression is the administration of a live vector generally by spray, feed or water where an infecting effective amount of the live vector (e.g. virus or bacterium) is provided to the animal. Another form of viral expression system is a non-replicating virus vector which is capable of infecting a cell but not replicating therein. The non-replicating viral vector provides a means of introducing to the human or animal subject genetic material for transient expression therein. The mode of administering such a vector is the same as a live viral vector.

In one preferred embodiment, a ddRNAi expression construct may be introduced into the target cells in vitro or ex vivo and then subsequently placed into an animal to affect therapy, or administered directly to an organism, organ or cell by in vivo administration. Delivery by viral infection is a preferred method of delivery; however, any appropriate method of delivery of the ddRNAi expression construct may be employed. The vectors comprising the cassettes can be administered to a mammalian host using any convenient protocol, where a number of different such protocols are known in the art.

The most common transfection reagents are charged lipophilic compounds that are capable of crossing cell membranes. When these are complexed with a nucleic acid they can act to carry DNA across the cell membrane. A large number of such compounds are available commercially. Polyethylenimine (PEI) is a new class of transfection reagents, chemically distinct from the lipophilic compounds, that act in a similar fashion, but have the advantage they can also cross nuclear membranes. An example of such a reagent is ExGen 500 (Fermentas). A construct or synthetic gene according to the present invention may be packaged as a linear fragment within a synthetic liposome or micelle for delivery into the target cell.

Compositions may also be injected by microinjection or intramuscular jet injection (for example as described by Furth et al., Anal. Biochem., 205: 265-368, 1992). Another mode of administration includes expression vectors, with restriction sites strategically engineered so as to facilitate the insertion of the relevant nucleic acid sequences. Transcription cassettes are a similar method for introducing the genetic constructs and agents of the invention, and may be carried by a variety of vectors, such as plasmids, retroviruses and the like. Desirably, such vectors are able to be transiently or stably maintained in the cells for at least a day and preferably longer. Another route of administration is hydrodynamic in which an aqueous formulation of the naked genetic construct, agent or synthetic gene is prepared, usually with a DNase inhibitor, and administered to the vascular system of the animal.

The techniques for delivery of DNA and RNA constructs to animal cells described in U.S. Pat. Nos. 5,985,847 and 5,922,687 are also applicable. The entire contents of these two specifications are incorporated herein by reference.

Tissue culture cells can be transformed using electroporation. This is thought to produce transient pores in cell membranes, through which DNA move into cells. In addition, animal cells can be transformed chemically using reagents such as PEG or calcium phosphate.

The RNAi agents and constructs of the present invention may also be delivered transdermally using a range of patch, spray, iontophoric or poration based methodologies.

Iontophoresis is predicated on the ability of an electric current to cause charged particles to move. A pair of adjacent electrodes placed on the skin set up an electrical potential between the skin and the capillaries below. At the positive electrode, positively charged drug molecules are driven away from the skin's surface toward the capillaries. Conversely, negatively charged drug molecules would be forced through the skin at the negative electrode. Because the current can be literally switched on and off and modified, iontophoretic delivery enables rapid onset and offset, and drug delivery is highly controllable and programmable.

Poration technologies, use high-frequency pulses of energy, in a variety of forms (such as radio frequency radiation, laser, heat or sound) to temporarily disrupt the stratum corneum, the layer of skin that stops many drug molecules crossing into the bloodstream. It is important to note that unlike iontophoresis, the energy used in poration technologies is not used to transport the drug across the skin, but facilitates its movement. Poration provides a “window” through which drug substances can pass much more readily and rapidly than they would normally.

The RNAi agents and ddRNAi expression constructs can be formulated into preparations for injection or administration by dissolving, suspending or emulsifying them in appropriate, pharmaceutically acceptable carriers or diluents. Examples of such pharmaceutically acceptable carriers or diluents include an aqueous or nonaqueous solvent, such as oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Accordingly, in another aspect, the present invention contemplates a pharmaceutical composition comprising a siRNA wherein said siRNA comprises a nucleotide sequence which is at least 70% identical to at least part of a nucleotide sequence comprising a HAT or a derivative, ortholog or homolog thereof.

In yet another aspect, the present invention contemplates a pharmaceutical composition comprising a ddRNAi expression construct wherein said ddRNAi construct comprises one or more ddRNAi expression cassettes each comprising a ddRNAi targeting sequence comprising a nucleotide sequence which is at least 70% identical to at least part of a nucleotide sequence comprising a HAT or a derivative, ortholog or homolog thereof.

The methods and compositions of the invention involve the administration of an effective amount of the RNAi agent or ddRNAi constructs so as to achieve the intended or desired result in modulation of gene expression. This is generally measured phenotypically. It is envisaged that, in some cases, some degree of routine trial and error, as is well known in the pharmaceutical arts, may be necessary in order to determine the preferred or most effective amount to be administered.

In order to observe the desired trait in multicellular organisms such as animals, in particular those which are tissue-specific or organ-specific or developmentally-regulated, regeneration of a transformed cell carrying the synthetic genes and genetic constructs described herein into a whole organism will be required. Those skilled in the art will be aware that this means growing a whole organism from a transformed animal cell, a group of such cells, a tissue or organ. Standard methods for the regeneration of certain animals from isolated cells and tissues are known to those skilled in the art. Accordingly, a further aspect of the invention provides a cell, tissue, organ or organism comprising the synthetic genes and genetic constructs described herein.

As would be evident to one of skill in the art, in another aspect, the present invention extends to a method of genetic therapy for treating hair loss or unwanted or abberant hair growth in a vertebrate animal, said method comprising administering to said subject a genetic construct comprising at least one ddRNAi expression cassette which encodes an RNA molecule comprising a nucleotide sequence which is at least 70% identical to at least part of a nucleotide sequence comprising a HAT or a derivative, ortholog or homolog thereof and which delays, represses or otherwise reduces the expression of the HAT in said subject.

Reference herein to “genetic therapy” includes gene therapy. The genetic therapy contemplated by the present invention further includes somatic gene therapy whereby cells are removed, genetically modified and then replaced into an individual. The gene therapy may be transient or permanent. Preferably, the animal is a human.

In another aspect, the present invention further extends to genetically modified cells comprising a ddRNAi expression construct as described herein, or a genomically integrated form or part thereof. Preferably the cell is a mammalian cell, even more preferably the cell is a primate or rodent cell and most preferably the cell is a human or mouse cell.

Although the present invention is particularly directed to animal or human hair follicle-forming skin cells, the scope of the invention extends to any vertebrate animal cell.

Preferably the vertebrate animal cells are derived from mammals, avian species, fish or reptiles. Preferably, the vertebrate animal cells are derived from mammals. Mammalian cells may be from a human, primate, livestock animal (e.g. sheep, cow, goat, pig, donkey, horse), laboratory test animal (e.g. rat, mouse, rabbit, guinea pig, hamster), companion animal (e.g. dog, cat) or captured wild animal. Particularly preferred mammalian cells are from human and murine animals.

Furthermore, in another aspect, the present invention contemplates a multicellular structure comprising one or more genetically modified cells described supra. The term “multicellular structure” should be understood to include a tissue, organ or complete organism comprising one or more of the genetically modified cells of the present invention. In one preferred embodiment, the present invention provides a genetically modified animal useful as an animal model. Such animal models are readily generated by grafting, for example, hair follicle-producing cells onto the skin of an animal such as a mouse. Nude mice are particularly useful in this regard as recipients of human hair follicle-producing cells. Such animals are useful test models for the genetic agents of the present invention.

The present invention is further described by the following non-limiting examples:

### EXAMPLE 1

### Development of an AA V-2 Expression Vector for in vivo Delivery of shRNA Sequences

Before the delivery of shRNA by infectious particles is tested, the appropriate expression plasmid is constructed and validated. There are at least two characteristics that need to be optimized in the RNAi expression construct: 1) the construct must be efficiently packaged into progeny virion; and 2) the plasmid must provide high levels of shRNA expression. In addition, in order to test the various RNAi expression constructs, there must be a means of assessing transfection and transduction efficiency.

In one embodiment, AAV-2 vectors which have been gutted of rep and cap provide the backbone (hereinafter referred to as the rAAV vector) for the viral RNAi expression construct. This vector has been extensively employed in AAV studies and the requirements for efficient packaging are well understood. The U6 and H1 promoters are used for the expression of shRNA sequences, though there have been reports of vastly different levels of inhibition of an identical shRNA driven independently by each promoter. However, vector construction is such that promoters can be easily swapped if such variation is seen.

As with virtually any viral delivery system, the rAAV vector must meet certain size criteria in order to be packaged efficiently. In general, an rAAV vector must be 4300-4900 nucleotides in length (McCarty et al. Gene Ther. 8: 1248-1254, 2001). When the rAAV vector falls below the limit, a ‘stuffer’ fragment must be added (Muzyczka et al. Curr. Top. Microbiol. Immunol. 158: 970129, 1992). Alternatively, the rAAV RNAi expression cassette may comprise two or more [promoter-RNAi-terminator] components.

In the AAV vector embodiment described here, each [promoter-RNAi-terminator] component is approximately 400 nucleotides in length leaving ample room for the inclusion of multiple [promoter-RNAi-terminator] components per expression cassette. Alternatively, one or more selectable marker cassettes may be engineered into the rAAV multiple-promoter RNAi expression construct in order to assess the transfection efficiency of the rAAV expression construct as well as allow for quantification of transduction efficiency of target cells by the rAAV expression construct delivered via infectious particles.

The initial test expression construct drives expression of a shRNA sequence designed from sequences with demonstrated ability to inhibit luciferase activity from a reporter construct (See, Elbashir et al. Embo. J 20(23): 6877-6888, 2001). The elements of the shRNA component, including the promoter, shRNA and the terminator sequence, are short and are assembled independently de novo utilizing long, complementary oligonucleotides that are then cloned into a viral vector using multiple cloning sites. A commercially available expression plasmid that encodes for the production of luciferase functions as the reporter to verify the ability of the shRNA to downregulate the target sequences.

Although the shRNA against luciferase has been previously validated, the efficacy of rAAV-delivered shRNA is assessed in vitro prior to testing the construct in vivo. The test and reporter constructs are transfected into permissive cells utilizing standard techniques. An rAAV expression construct in which the luciferase-specific shRNA has been replaced by an unrelated shRNA sequence is utilized as a negative control in the experiments. The relative percentage of transfection efficiency is estimated directly by assessing the levels of the selective marker using fluorescence microscopy. For assessing inhibitory activity of the shRNA, luciferase activity is measured utilizing standard commercial kits. Alternatively, quantitative real time PCR analysis (Q-PCR) is run on RNA that is harvested and purified from parallel experimental plates. Activity decreases greater than 90% percent, relative to the activity recovered in lysates from cells treated with the unrelated shRNA species, are an indication that the shRNA is functional.

Once it is established that the expression construct is functional in both in vitro cell culture systems as well as in vivo models by utilizing co-transfection of the naked DNA plasmids, testing is initiated on the rAAV RNAi expression construct packaged into infectious particles. The infectious particles are produced from a commercially available AAV helper-free system that requires the co-transfection of three separate expression constructs containing 1) the rAAV construct expressing the shRNA against luciferase (flanked by the AAV ITRs); 2) the construct encoding the AAV rep and cap genes; and 3) an expression construct comprising the helper adenovirus genes required for the production of high titer virus. Following standard purification procedures, the viral particles are ready for use in experiments.

### EXAMPLE 2

### Development of an rAA V Expression Construct

Construction of a RNAi expression cassette includes promoter and terminator sequences that drive expression of the ddRNAi agent at a therapeutic level. The synthesis of small nuclear RNAs and transfer RNAs is directed by RNA polymerase III (pol III) under the control of pol III-specific promoters. Because of the relatively high abundance of transcripts directed by these regulatory elements, pol III promoters, including those derived from the U6 and H1 genes, have been used to drive the expression of shRNA (see, eg., Domitrovich and Kunkel. Nucl. Acids Res. 31(9): 2344-52, 2003); Boden et al. Nucl. Acids Res. 31(17): 5033-38, 2003a; and Kawasaki et al. Nucleic Acids Res. 31(2): 700-7, 2003).

Initially, the assessment of relative promoter strength of the pol III-specific sequences is conducted in vectors containing the individual promoters. Each promoter construct drives expression of the same shRNA with demonstrated functional inhibition of luciferase activity (Elbashir et al. 2001, supra). Since there is a wealth of data demonstrating the successful utilization of the U6 promoter for the expression of shRNA, it is used as the standard for assessing the relative strength of other promoters. The majority of the promoters that are tested are quite short, most in the range of 200-300 nucleotides in length. Long, overlapping oligonucleotides are used to assemble the promoters and terminators de novo and are then cloned into multiple cloning sites that flank the shRNA. The promoter is paired with the termination signal that occurs naturally downstream of the gene from which the promoter is taken.

The relative strength of each promoter is assessed in vitro by the decrease in activity of a co-transfected luciferase reporter. The test and reporter constructs are transfected into permissive cells utilizing standard techniques. Controls consist of a test promoter construct in which the functional shRNA against luciferase is replaced by an unrelated shRNA sequence. A third marker construct encoding for the secreted protein human α1-antitrypsin (hAAT) is co-transfected into the cells in order to assess for variations in transfection efficiencies. For assessing inhibitory activity of the shRNA, luciferase activity is measured utilizing standard commercial kits. The shRNA-mediated decrease in luciferase expression, normalized to hAAT levels, is an indirect measurement of promoter strength. Alternatively or in addition, quantitative real time PCR analysis (Q-PCR) on luciferase RNA levels is performed on RNA that is harvested and purified from parallel experimental plates.

Once appropriate promoter and terminator pairs are identified, a multiple-promoter RNAi expression cassette may be designed. Several designs of the final vector are tested, including having all three [promoter-RNAi-terminator] components in a tandem array or arranged in clockwise and counterclockwise configurations (i.e., transcribed from the top and the bottom strand of the cassette DNA) or any variation thereof, such as shown in FIGS. 3A and 3B.

Each configuration is transfected into cells and tested for inhibitory activity utilizing luciferase activity assays. Two or more promoters driving distinct ddRNAi agents may result in an additive or synergistic inhibitory effect, thus, in order to assess the functionality and relative strength of each of the promoters within the context of the a multiple-promoter expression construct, variants of the expression cassettes are generated. Utilizing these ddRNAi agents, the inhibitory effect of the shRNA driven from each promoter within the multiple-expression construct is measured by luciferase assays. Alternatively, Q-PCR may be used to assess relative levels of transcript driven by each promoter. Although the self-complementary nature of hairpin-RNA generally would prevent the direct Q-PCR measurement of these RNA transcripts, different non-hairpin transcripts of approximately the same size can be substituted into the vectors in place of the shRNA, or viral multiple-promoter RNAi expression constructs with cassettes such as those shown in FIGS. 3C and 3D may be used.

### EXAMPLE 3

### Selection and Testing of ddRNAi Agents for Modulation of Hair Growth

The selection of shRNAs useful as agents for the modulation of hair growth is not a straight-forward proposition. The first step is the selection of the target gene. For example in the case of the DHT receptor gene, to select candidate sequences, an alignment of all published independent full-length or near-full-length DHT receptor encoding sequences is performed. When the sequence analyses are concluded, a list of candidate RNAi sequences is generated. In order to rank the sequences on the basis of relative potency, the ability of individual pre-synthesized RNAi agents to inhibit the activity of the DHT receptor gene is tested.

Specifically, pre-synthesized RNAi agents are transfected into tissue such as follicular skin tissue by standard techniques and reagents. An unrelated RNAi species is transfected into a parallel set of plates to serve as the negative control. Transfection efficiency is monitored by the inclusion of a small amount of non-specific RNAi into the transfection mixture that is end-labeled with fluorescein or phycoerythrin. The relative transfection efficiency is gauged by fluorescence microscopy prior to analysis of down regulation efficacy. At various time points post-transfection, the level of the DHT receptor gene activity is measured by one of a variety of methods, such as the abundance of the DHT receptor in the subject tissue.

Highly functional ddRNAi agents are selected and tested individually, and are then transfected into cells in multiples. One control consists of transfecting an equivalent number of unrelated ddRNAi agents in parallel. The inhibitory activity of the multiple transfections is compared to activity from a set of parallel plates that have been transfected with only one RNAi agent.

In embodiments where multiple RNAi agents are used, RNAi agents are validated, and the coding sequences for each corresponding shRNA is generated from long, complementary self-annealing oligonucleotides and cloned into the individual sites of the multiple promoter cassette. This cassette is then inserted into a viral vector and this construct is then packaged into viral particles according to the methods described herein. The total length of each [promoter-RNAi-terminator] component of the RNAi expression cassette is small (˜400 nucleotides); linking three [promoter-RNAi-terminator] components together results in a sequence that is 1200-1300 nucleotides in length, far below the upper size limit of self-complementary AAV.

The inhibitory activity of the viral particles is tested on the subject cells or tissue (such as follicular skin tissue). Generation of a multiple-promoter construct expressing unrelated shRNA species serves as a negative control. The efficacy of the shRNA sequences is monitored by aforementioned analysis techniques.

While the present invention has been described with reference to specific embodiments, it should be understood by those skilled in the art that various changes may be made and equivalents may be substituted without departing from the true spirit and scope of the invention. In addition, many modifications may be made to adapt a particular situation, material or process to the objective, spirit and scope of the present invention. All such modifications are intended to be within the scope of the invention.

### BIBLIOGRAPHY

Ahmad et al., Science 279: 720-724, 1998;
Boden et al., J. Virol. 77(21): 115231-35, 2003;
Boden et al. Nucl. Acids Res. 31(17): 5033-38, 2003;
Chen et al., Molecular Therapy 8(3): 495-500, 2003;
Domitrovich and Kunkel. Nucl. Acids Res. 31(9): 2344-52, 2003;
Elbashir et al. Nature 411: 494-498, 2001;
Elbashir et al Embo. J. 20(23): 6877-6888, 2001;
Fire et al, Nature 391: 806-11, 1998;
Furth et al., Anal. Biochem., 205: 265-368, 1992;
Grimm et al. Blood. 2003-02-0495;
Harborth et al., Antisense Nucleic Acid Drug Rev. 13(2): 83-105, 2003;
Higashibata et al., J. Bone Miner. Res. 19(1): 78-88, 2004;
Hoggatt et al., Circ. Res. 91(12): 1151-59, 2002;
Karlin and Altschul, Proc. Natl Acad. Sci. USA 87: 2264-2268, 1990;
Kawasaki et al. Nucleic Acids Res. 31(2): 700-7, 2003;
Kay et al., Nature 424: 251, 2003;
Kljuic et al., Cell 113: 249-260, 2003;
Kurreck, Eur. J. Bioch. 270: 1628-44, 2003;
Maraia et al., Nucleic Acids Res 22(15): 3045-52, 1994;
Maraia et al., Nucleic Acids Res 24(18): 3552-59, 1994;
McCarty et al. Gene Ther. 8: 1248-1254, 2001;
Mingozzi et al., J. Virol. 76(20): 10497-502, 2002;
Morris et al., Nature Biotechnology 22(4): 411-417;
Muzyczka et al. Curr. Top. Microbiol. Immunol. 158: 970129, 1992;
Myers and Miller, Comput. Appl. Biosci. 4:11-17, 1988;
Nakai et al., J. Virol. 76: 11343-349, 2002;
Pearson and Lipman, Proc Natl Acad Sci USA. 85(8): 2444-8, 1988;
Perri et al, J. Virol. 74(20): 9802-07, 2002;
Sambrook et al., Molecular Cloning: A Laboratory Manual, 2nd Ed., Cold Spring Harbor Press, Cold Spring Harbor, N.Y. (1989)
Sato et al., J. Clin. Invest. 104: 855-864, 1999;
Sohal et al., Circ. Res. 89(1): 20-25, 2001;
Thomas et al., Nature Reviews, Genetics 4: 346-58, 2003;
Tomar et al., Oncogene 22: 5712-15, 2003;
Tuschl et al. Genes and Dev. 13: 3191-97, 1999;
U.S. Pat. No. 5,422,262;
U.S. Pat. No. 5,922,687;
U.S. Pat. No. 5,985,847;
U.S. Pat. No. 6,573,099;
U.S. Pat. No. 6,620,805;
U.S. Pat. No. 6,673,611;
U.S. Pat. No. 6,773,881;
US Patent Application Publication No. 2004/0147022;
US Patent Application Publication No. 2004/0147023;
US Patent Application Publication No. 2004/0147470;
US Patent Application Publication No. 2004/0161777;
US Patent Application Publication No. 2004/0161844;
US Patent Application Publication No. 2002/0162126;
US Patent Application Publication No. 2004/0171030;
Us Patent Application Publication No. 2004/0171031;
Us Patent Application Publication No. 2004/0171032;
US Patent Application Publication No. 2004/0171570;
US Patent Application Publication No. 2004/0171579;
WO 03/022052 A1;
Xia et al, Nucleic Acids Res 31-17, 2003;
Yant et al., Nature Biotech. 20: 999-1004, 2002;
Zhang et al., Genome Res. 14(1): 79-89, 2004.

What is claimed is:

1. A method for modulating the growth, development, or maintenance of hair or hair follicles in a vertebrate animal subject, said method comprising administering to said subject an RNAi agent, wherein said RNAi agent delays, represses or otherwise reduces the expression of a hair associated genetic target in the vertebrate animal subject.

2. The method of claim 1, wherein the RNAi agent comprises a nucleotide sequence which is at least 70% identical to at least part of a nucleotide sequence comprising a hair associated genetic target, or a derivative, ortholog, or homolog thereof.

3. The method of claim 1, wherein the hair associated genetic target comprises a nucleotide sequence encoding 5α-dihydrotestosterone (DHT) receptor, steroid 5α-reductase polypeptide 1, or steroid 5α-reductase polypeptide 2.

4. The method of claim 1, wherein the hair associated genetic target comprises a nucleotide sequence encoding the hairless (hr), the lanceolate hair (lah) locus, Dsg4 (Desmoglein 4), Shh (Sonic hedgehog), Vegf, Cd34 (Cd34 antigen), S100, Ibd2 (Ibd2 helix-loop-helix antagonist), Ibd4, Peg3 (Paternally expressed gene 3), Fzd2 (Frizzled 2), Dkk3 (Dickkopf homolog 3), Sfrp1 (Secreted Frizzled Related Protein 1), Dab2 (Disabled homolog 2), Cktsflb1 (Gremlin, cysteine knot superfamily 1, BMP antagonist 1), Fgfr1 (Fibroblast growth factor receptor 1), Fgt1 (Fibroblast growth factor 1), Gpr49 (G-protein-coupled receptor 49), Igfbp5 (Insulin-linke growth factor binding protein 5), Myoc (Trabecular meshwork induced glucocorticoid protein), Itm2a (Integral membrane protein 2A), Eps8 (Epidermal growth factor receptor pathway substrate 8), Fyn (Fyn proto-oncogene), Col6a1 (Procollagen, type IV, alpha 1), Tnc (Tenascin C), Krt2-6a (Keratin complex 2, basic, gene 6a), Potassium channel subfamily K encoding sequences, Skd3 (Suppressor of K+ transport defect 3), Clic4 (Chloride intracellular channel 4), Col18al (Endostatin, alpha 1 (XVIII) collagen), Gna14 (Guanine nucleotide binding protein), Ly6 (Lymphocyte antigen 6 complex), Bmp4 (Bone morphogenetic protein 4), II1r2 (Interleukin 1 receptor, type II), Wnt3a (Wingless-related MMTV integration site 3A), II12rb2 (Interleukin 12 receptor, beta 2), Wnt10a (Wingless-related MMTV integration site 10a), Ifngr2 (Interferon-gamma receptor precursor), Fgfbp1 (Fibroblast growth factor binding protein 1), Klf5 (Kruppel-like factor 5), Gata3 (GATA binding protein 3), Retinoic acid stimulated basic helix-loop-helix protein encoding sequences, Mki67 (antigen identified by monoclonal antibody Ki-67), Cks2 (CDC28 protein kinase regulatory subunit 2), Ccng2 (Cyclin G2), or Prc1 (Protein regulator of cytokinesis 1).

5. The method of claim 1, wherein administering an RNAi agent comprises administering a ddRNAi expression vector comprising one or more ddRNAi expression cassettes that effect RNAi-mediated silencing of one or more hair associated genetic targets.

6. The method of claim 5, wherein the ddRNAi expression vector comprises at least two ddRNAi expression cassettes.

7. The method of claim 5, wherein the ddRNAi expression vector is administered to the vertebrate animal subject by a viral delivery system.

8. The method of claim 7, wherein the viral delivery system is an Adeno-Associated Virus (AAV) derived delivery system.

9. A ddRNAi expression vector comprising one or more ddRNAi expression cassettes, wherein each ddRNAi expression cassette, once expressed in a host cell effects transcription of an RNAi agent which effects RNAi-mediated silencing of a hair associated genetic target.

10. A pharmaceutical composition comprising:
a ddRNAi expression vector comprising one or more ddRNAi expression cassettes, wherein each ddRNAi expression cassette, once expressed in a host cell effects transcription of an RNAi agent which effects RNAi-mediated silencing of a hair associated genetic target; and
a pharmaceutically acceptable carrier or diluent.
